(19) United States
(12) Patent Application Publication
(10) Pub. No.: US 2026/0151202 A1
(43) Pub. Date: Jun. 4, 2026

(54) SYSTEMS, DEVICES, AND METHODS FOR HUMANOID SURGICAL ROBOT TRAINING AND USE

(71) Applicant: Howmedica Osteonics Corp, Mahwah, NJ (US)

(72) Inventors: Kevin ABBRUZZESE, Mahwah, NJ (US); Mark CASS, Redwood City, CA (US); Sina POURGHODRAT, Fort Lauderdale, FL (US)

(73) Assignee: Howmedica Osteonics Corp, Mahwah, NJ (US)

(21) Appl. No.: 19/408,815

(22) Filed: Dec. 4, 2025

Related U.S. Application Data
(60) Provisional application No. 63/727,908, filed on Dec. 4, 2024.

(51) Int. Cl. A61B 34/32; A61B 34/00; A61B 34/10; A61B 34/20

(52) U.S. Cl. CPC A61B 34/32; A61B 34/10; A61B 34/20; A61B 34/25; A61B 2034/101; A61B 2034/107; A61B 2034/2048

(57) ABSTRACT

According to an example, a method for gathering surgical data may include programming a humanoid robot to perform a task. The humanoid robot may include one or more measurement devices coupled to the humanoid robot. The method may further include receiving surgical data associated with the one or more measurement devices during a performance of the task by the humanoid robot.

### CROSS-REFERENCE TO RELATED APPLICATION(S)

This patent application claims the benefit of priority to U.S. Provisional Application No. 63/727,908, filed on Dec. 4, 2024, the entirety of which is incorporated herein by reference.

### FIELD OF THE DISCLOSURE

This disclosure relates to performing medical procedures with humanoid robots, among other aspects. In particular, this disclosure relates to devices and methods for using and training humanoid robots and for tuning humanoid robots in order to enhance surgical access and to improve surgical outcomes after one or more medical procedures, such as a joint replacement procedure, among other aspects.

### BACKGROUND OF THE DISCLOSURE

Conventional surgical techniques and procedures (for example, a joint replacement procedure) generally require a surgeon to be physically present in an operating room. The conventional solution to this requirement are robots telemanipulated by surgeons. The conventional approach generally requires significant resource investment in facilities to support existing telemanipulated surgical robot platforms. This may result in reduced access to critical surgical procedures, such as orthopedic procedures, in places without the requisite surgeons or conventional telemanipulated robots. Autonomous humanoid robots may address these access limitations, but conventional approaches to training humanoid robots to perform effectively have been limited.

Improved systems, devices, and methods for training and use of telemanipulated, semi-autonomous, and autonomous humanoid surgical robots are thus desired.

### BRIEF SUMMARY OF THE DISCLOSURE

Aspects of the disclosure relate to, among other things, systems, devices, and methods of using and training humanoid robots and for tuning humanoid robots in order to enhance surgical access and to improve surgical outcomes after one or more medical procedures, such as a joint replacement procedure, among other aspects. Each of the aspects disclosed herein may include one or more of the features described in connection with any of the other disclosed aspects.

According to an example, a method for gathering surgical data may include programming a humanoid robot to perform a task. The humanoid robot may include one or more measurement devices coupled to the humanoid robot. The method may further include receiving surgical data associated with the one or more measurement devices during a performance of the task by the humanoid robot.

Any of the systems, devices, and methods described herein may include any of the following features. The humanoid robot may include a surgical phenotype selection module. The method may include determining a surgical phenotype of the humanoid robot based on the surgical phenotype selection module. The surgical phenotype selection module may include a user model, and the humanoid robot may include one or more electromechanical systems. The method may further include actuating the one or more electromechanical systems to conform to the surgical phenotype determined by the surgical phenotype selection module. The surgical phenotype may be determined based on the user model. The user model may be a trained machine learning model. The surgical phenotype selection module may include a surgical model configured to receive surgical data including at least one movement of a human surgeon performed during a surgery, and programming the humanoid robot to perform the task may be based on an output from the surgical model. The surgical model may be a trained machine learning model. The method may further include receiving surgical phenotype data from one or more data sources, the one or more data sources may include prior surgical data of one or more patients with at least one attribute in common with a current patient and determining the surgical phenotype of the humanoid robot based on the phenotype data. The one or more data sources may include at least one of preoperative data relating to one or more patient, postoperative data relating to one or more patients, and intraoperative data. The method may further include determining intraoperative outputs based on intraoperative data, the intraoperative outputs including one or more of a difference between an actual measurement and an expected measurement associated with the task or a difference between a motion of a surgeon and a corresponding motion by the humanoid robot. The intraoperative data may be received from one or more sensors and the one or more sensors may include at least one of a force sensor, a displacement sensor, a pressure sensor, and an imaging device.

According to another example, a system for gathering test data for one or more medical devices may include a humanoid robot including one or more processors, one or more measurement devices coupled to the humanoid robot, and one or more sensors configured to gather intraoperative data associated with the one or more measurement devices during a performance of a programmed task by the humanoid robot.

Any of the systems, devices, and methods described herein may include any of the following features. The humanoid robot may include a surgical phenotype selection module configured to determine a surgical phenotype of the humanoid robot. The surgical phenotype selection module may include a surgical model configured to receive surgical data including at least one movement of a human surgeon performed during a surgery, and the humanoid robot may include one or more electromechanical systems. Parameters of the one or more electromechanical systems may be defined based on the surgical phenotype selection module. The surgical model may be a trained machine learning model. The surgical phenotype selection module may include a surgical model, and programming the humanoid robot to perform the task may be based on output from the surgical model. The surgical model may be a trained machine learning model. The system may include one or more data sources including surgical phenotype data. The surgical phenotype data may include both surgical model data and user model data. Determining the surgical phenotype of the humanoid robot may be based on the surgical phenotype data. The one or more data sources may include at least one of preoperative data relating to one or more patients, postoperative data relating to one or patients, or intraoperative data. The system may be further configured to determine intraoperative outputs based on the intraoperative data, the intraoperative outputs may include one or more of a difference between an actual measurement and an expected measurement associated with the task, or a difference between a motion of a surgeon and a corresponding motion by the humanoid robot.

According to another example, a system may include a programmable humanoid robot including one or more measurement sensors. The programmable humanoid robot may include at least one processor and at least one storage medium including instructions which, when executed by the at least one processor, cause the at least one processor to perform operations including receive surgical phenotype data from one or more data sources, determine, using a surgical phenotype selection module, a surgical phenotype of the humanoid robot based on the surgical phenotype data, adjust one or more electromechanical systems to conform the humanoid robot to the surgical phenotype determined by the surgical phenotype selection module, program the humanoid robot to perform a task based on a surgical model, receive intraoperative data associated with the one or more measurement sensors during a performance of the task by the humanoid robot, and causing to display, on one or more display devices, intraoperative outputs based on the intraoperative data associated with the one or more measurement sensors.

According to another example, a computer-implemented method for generating training data for training a humanoid robot to perform a task may include capturing, by one or more processors, a video recording of a user performing the task, the video recording including: image data of at least one of hand positions and arm positions of the user performing the task, the image data comprising one or more video frames, and time stamp data associated with the image data, with each of the one or more video frames comprising a respective time stamp; based on the image data and the time stamp data, generating, by the one or more processors, recorded trajectory data of the user performing the task; transforming, by the one or more processors, the recorded trajectory data into predicted robot trajectory data based on a kinematical model of a robot; generating, by the one or more processors, a robot experience training video, by: (i) digitally removing the user from each of the one or more video frames; and (ii) based on the predicted robot trajectory, inserting the kinematic model of the robot into each of the one or more frames; and outputting, by the one or more processors, the robot experience training recording to a programmable humanoid robot.

In another example, a robotic training system may include: a data capture module configured to capture a video recording of a user performing a task, the video recording including image data of at least one of hand positions and arm positions of the user and time stamp data associated with the image data; a trajectory generation module configured to generate recorded trajectory data of the user performing the task based on the image data and the time stamp data; a transformation module configured to transform the recorded trajectory data into predicted robot trajectory data based on a kinematical model of a robot; a video processing module configured to generate a robot experience training video by digitally removing the user from video frames and inserting the kinematic model of the robot into the video frames based on the predicted robot trajectory; and an output module configured to output the robot experience training video to a programmable humanoid robot.

In yet another example, a method of operating a trained humanoid robot to perform a task may include: capturing, by a sensor system mounted on the robot, current environmental data including RGB camera data and inertial measurement unit data; inputting, by one or more processors, the current environmental data and predefined trajectory points to a trained machine learning model; receiving, by the one or more processors, from the trained machine learning model, predicted trajectory data for robot movement and a predicted RGB frame; generating, by the one or more processors, robot motion commands based on the predicted trajectory data; executing, by the robot, the robot motion commands to move toward completion of the task; and performing, by the one or more processors, consistency checking by comparing the predicted RGB frame with subsequently captured RGB camera data.

### BRIEF DESCRIPTION OF THE DRAWINGS

A more complete appreciation of the subject matter of this disclosure and the various advantages thereof may be realized by reference to the following detailed description, in which reference is made to the following accompanying drawings:

FIG. 1 is an exemplary testing environment for surgical humanoid robots, according to aspects of this disclosure.

FIG. 2 is an exploded illustration of a humanoid robot with a plurality of electromechanical systems, according to aspects of this disclosure.

FIG. 3 is an illustration of an electromechanical of a humanoid robot limb, according to aspects of this disclosure.

FIG. 4 illustrates a system for collection, processing, transmission, and storage of intraoperative data associated with an operative patient, according to aspects of this disclosure.

FIG. 5 is a schematic diagram depicting the processing of preoperative, intraoperative, and postoperative data and outputs of the system of FIG. 4, according to aspects of this disclosure.

FIG. 6 is a schematic diagram exemplifying types of postoperative data and outputs of the system of FIG. 4, according to aspects of this disclosure.

FIG. 7 illustrates an exemplary testing and simulation diagram for surgical phenotype selection, according to aspects of this disclosure.

FIG. 8 shows an exemplary machine learning training flow chart, according to aspects of this disclosure.

FIG. 9 is a functional block diagram of a computer, according to aspects of this disclosure.

FIG. 10 depicts an exemplary training environment 400 for training and using humanoid robots, according to aspects of this disclosure,

FIG. 11 is a flow chart showing a method for generating training data for training a humanoid robot to perform a task.

FIG. 12 is a flow chart showing a method of operating a trained humanoid robot to perform a task.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

In this disclosure, “user'” is synonymous with “practitioner'” and may be any person completing the described action (e.g., surgeon, technician, nurse, etc.). An implant may be a medical device that is at least partially implanted in a patient and/or provided inside of a patient's body. For example, an implant may be a sensor, artificial bone(s), or other medical device coupled to, implanted in, or at least partially implanted in a bone, skin, tissue, organs, etc. A prosthesis or prosthetic may be a device configured to assist or replace a limb, bone, skin, tissue, etc. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense. For example, a prostheses may be coupled to a patient at an exterior portion of the patient's body, such as a knee brace or other device coupled to an exterior portion of the patient.

As used herein, the term “autonomous” may refer to a humanoid robot capable of performing tasks independently without direct human control during execution, and may include semi-autonomous operation where the robot performs certain tasks independently while receiving intermittent human guidance or oversight. As used herein, the term “telemanipulated” may refer to a robotic system that is controlled remotely by a human operator who directs the robot's movements and actions in real-time through a control interface.

Medical devices discussed in this disclosure may include, for example, implants, prosthetics, or wearable items.

In describing embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

It will also be understood that, although the terms first, second, third, etc. are, in some instances, used herein to describe various elements, these elements should not be limited by these terms. These terms are only used to distinguish one element from another. For example, a first input could be termed a second input, and, similarly, a second input could be termed a first input, without departing from the scope of the various described embodiments. The first input and the second input are both inputs, but they are not the same input.

Implementation of a humanoid robot for surgical procedures may provide numerous clinical benefits. Humanoid robots may be deployed in areas where deployment of conventional telemanipulated robots is impractical or impossible. Humanoid robots may have enhanced precision, dexterity, and endurance as compared to human surgeons. Humanoid robots may, in some instances, improve patient outcomes. Humanoid robots may be telemanipulated, and through AI and ML systems, may learn and mimic surgeon movements, techniques, and preferences via real-time feedback, which may ultimately enable partial to full autonomy. While reference is made herein to use of robotic humanoid robots in the surgical context, the systems, devices, and methods disclosed herein may be applicable to a wide range of medical applications, including patient consultations, follow-up appointments, rehabilitation, etc. Additionally, one of ordinary skill in the art will appreciate that the systems, devices, and methods of the disclosure may be applied to various non-medical use cases and scenarios.

Humanoid robots may enhance one or more workflows or tasks typically performed by human surgeons. AI systems associated with the humanoid robots may reduce the mental and physical demands placed on human surgeons. The AI systems may support clinical decision making by providing insights from data generated during procedures, which may reduce analytical uncertainty regarding patient outcomes. The surgical humanoid robot may be adapted to surgeon preferences to continuously train during procedure and may execute portions of procedures autonomously or under guidance. A humanoid robot may assess surgical procedures to provide additional insights and measurements via sensors such as cameras (including optical, LIDAR, and/or infrared), IMUs, and force sensors to provide objective information that may not be captured by conventional surgical tools. Additional sensors may include, but are not limited to, encoders (rotary and/or linear), magnetometers, strain gauges, torque sensors, load cells, capacitive touch sensors, piezoelectric sensors, resistive pressure sensors, ultrasonic sensors, LiDar (lght detection and ranging) sensors, and/or time-of-flight sensors. Vision systems of the humanoid robot may include monocular, stereo, depth, and/or thermal cameras, The humanoid robot may include temperature sensors, humidity sensors, barometric pressure sensors, microphones (including directional microphones), heart rate sensors, pulse oximeter sensors, gas sensors (including carbon dioxide and/or oxygen), geolocation (e.g., GPS, GLONASS, etc.), RFID and/or Bluetooth sensors. Any of the sensors of the humanoid robot may correspond with any portion, component, element, or system of the humanoid robot.

Humanoid surgical robots are bipedal robotic platforms designed to navigate and understand complex surgical environments. Humanoid surgical robots may move autonomously, stepping over or maneuvering around obstacles with mobility and awareness comparable to a human surgeon. Humanoid robots are ambidextrous and are capable of executing tasks with both hands simultaneously, with each hand performing with identical accuracy and precision. In an example, a humanoid robot may utilize one hand to perform a surgical task while using the other hand to stabilize a patient's anatomy or to provide error correction. In an example, the non-task hand, equipped with encoders and sensors, may detect unintended patient movement. This feedback may be processed through a machine learning (ML) algorithm, such as a Kalman Filter, or a more sophisticated brain physiology-inspired bilateral neural network, to enable fine motor control and immediate corrective action for a given surgical task.

Humanoid robots are equipped to generate real-time, digital twin models of a patient's anatomy through advanced scanning and vision systems. These humanoid robots may locate and scan the patient's body with tactile sensors in their hands, thereby generating an organized point cloud of the patient anatomy. Using this data, a digital twin mesh of the patient may be constructed through surface reconstruction algorithms, or alternatively, by training a Generative Adversarial Neural Network (GAN) to produce the mesh directly from the point cloud data. Humanoid robots may integrate multiple preoperative imaging modalities as inputs, receiving data from sources such as CT scans, MRI, X-rays, and ultrasound to accurately map the digital twin with physical assessments, thereby enabling more accurate surgical planning and outcome prediction.

In addition to its sensory capabilities, a humanoid robot may also wear traditional human surgical equipment, such as gloves and gowns, and may interact with medical devices designed for human operation, making them suitable for sterile environments and human-centered equipment.

The development of the underlying software of humanoid robots relies on previously trained machine learning models. Transfer Learning, a technique utilizing pre-trained models as the foundation for new ones, may allow for efficient adaptation and continuous improvement in robotic capabilities. These models may be stored in permanent storage of the robot, for example, on an SSD. Continuous data collection from the humanoid robot's sensors, however, may quickly exhaust storage resources. To manage this, ML algorithms with “Out of Distribution (OOD)” detection capabilities may discern and retain only relevant data, optimizing storage usage while preserving essential information for future model training and refinement.

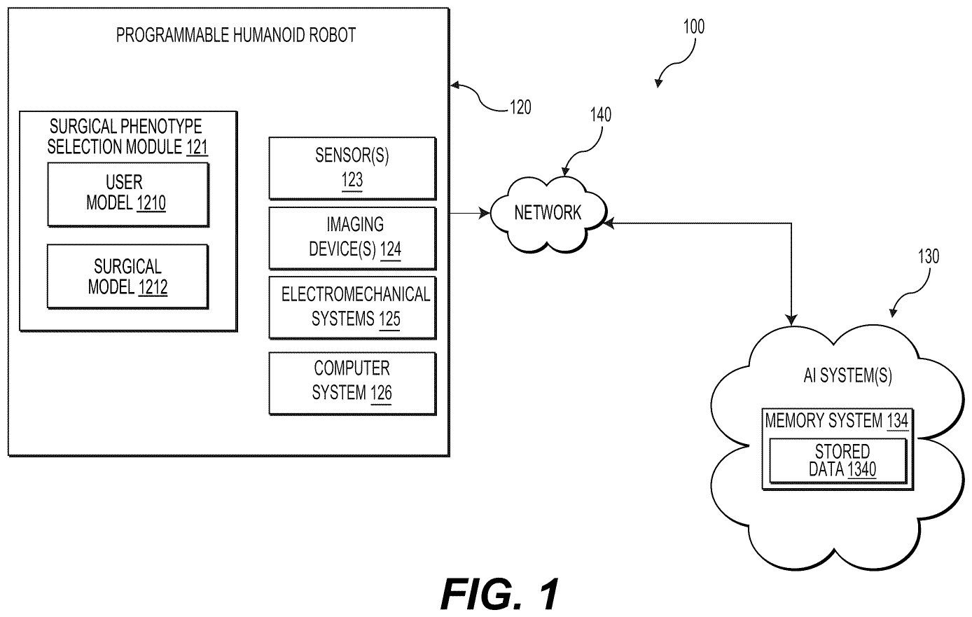

The surgical humanoid robot may enhance surgical workflows and execute surgeon guided decisions without physiological limitations, fatigue, stress, or cognitive demand. The surgical humanoid robot may also observe the aforementioned factors of humans in the operating room and provide suggestions accordingly (e.g., providing notifications when observing a fatigued user). Surgical techniques and data gathering may be performed on a programmable humanoid robot 120, shown diagrammatically in FIG. 1.

FIG. 1 depicts an exemplary environment for use of surgical humanoid robots, according to aspects of this disclosure. One or more components of the environment 100 may communicate with one or more of the other components of the environment 100 across electronic network 140, including one or more systems and elements associated with a programmable humanoid robot 120, and one or more systems and elements associated with AI systems 130, which may be stored within a cloud, where cloud may be any local or networked system suitable for transferring data. A central server associated with AI System(s) 130 may be used to manage and synchronize content associated with humanoid robot 120, ensuring consistent and up-to-date information is transmitted to humanoid robot 120. The central server may include a central content management system, using a high-level web framework to manage data stored in memory system 134, with content delivery optimized through a content delivery network. Additionally, while a single (e.g., only one) humanoid robot 120 is depicted in FIG. 1, it is understood that environment 100 may include a plurality of humanoid robots 120 (e.g., a network of humanoid robots) without departing from the scope of this disclosure. While discussion below primarily describes orthopedic surgery, it will be understood that the systems, devices, and methods of the disclosure may be applicable to various other types of surgery based on the techniques described herein.

Programmable humanoid robot 120 may include a surgical phenotype selection module 121. Surgical phenotype selection module 121 may be used to tune one or more systems (including hardware and/or software) and/or components of the humanoid robot 120, to a specific surgeon to perform one more tasks associated with a surgical procedure according various types of information associated with the surgeon. Surgical phenotype selection module 121 may include specific attributes, characteristics, and/or techniques for a given surgeon. Adjustment of a surgical phenotype may allow multiple surgeons to a use a same programmable humanoid robot 120 in a familiar and individualized manner.

Surgical phenotype selection module 121 may be tuned to accommodate different users, such as surgeons, based on their physical statures and other characteristics. For example, the module may adjust various parameters of humanoid robot 120 to match the physical attributes and working preferences of different surgeons. In some cases, a short female surgeon may prefer certain ergonomic configurations, such as a lower operating table height, closer positioning to the surgical site, and specific arm reach parameters that correspond to her stature. The phenotype selection module 121 may adjust the humanoid robot's joint angles, reach distances, and positioning strategies to replicate these preferences. Conversely, a tall male surgeon may work with different spatial relationships, including a higher table position, greater reach distances, and different viewing angles. The phenotype selection module 121 may modify the kinematic parameters of the humanoid robot 120, including shoulder height, arm extension ranges, and torso positioning, to accommodate these differences. The phenotype selection module 121 may also account for differences in hand size, grip strength, and manipulation patterns that may correlate with different body types, thereby enabling the humanoid robot 120 to perform surgical tasks in a manner that reflects the natural working style of surgeons with varying physical characteristics.

Surgical phenotype selection module 121 may include at least a user model 1210 and a surgical model 1212. In some examples, user model 1210 and surgical model 1212 may be incorporated into a single phenotype selection model. Programmable humanoid robot 120 may include one or more sensors 123, which may include, for example, inertial measurement units (IMUs), strain gauges, accelerometers, gyroscopes, temperature sensors, one or more imaging devices 124, for example cameras or infrared devices, one or more electromechanical systems 125, which may include, for example, viscoeleastic materials, piezoelectric materials, elastic materials, etc., that may be driven by, controlled by, manipulated by or actuated by motors, hydraulic and/or pneumatic systems, linkages, etc. to mimic or simulate bone, muscle, or other soft tissues, such as, for example, tendons, ligaments, etc., and additional computer systems 126 for operating any of the elements recited.

User model 1210 may be a trained machine learning model stored locally within surgical phenotype selection module 121, stored in computer system 126, or may be stored in stored data 1340 of a memory system 134 associated with one or more AI system(s) 130. The term “machine learning model” may generally encompass instructions, data, and/or a model configured to receive input, and apply one or more of a weight, bias, classification, or analysis on the input to generate an output. The output may include, e.g., a classification of the input, an analysis based on the input, a design, process, prediction, or recommendation associated with the input, or any other suitable type of output.

The inputs to user model 1210 may include, for example, information related to one or more users (e.g., surgeons) that may use humanoid robot 120 to perform a surgical procedure. The information may be associated with a user from a user profile, for example. The information may be related to preferred surgical techniques and/or surgical approaches/methodologies. The inputs may include information indicating that a given user may specialize in a specific type of surgery (e.g., joint reconstruction, orthopedic oncology, sports medicine, pediatric orthopedics, etc.). The inputs may include a preferred alignment approach, which may refer to the preferred strategies the user applies in aligning a joint. Preferred alignment approaches may include for example: individualized (e.g., alignment catered to a given patient's anatomy), mechanical (e.g., standardized, neutral alignment), and customized (e.g., incorporating aspects of both individualized and mechanical alignment). The inputs may include a user type, which may include robotic users and manual users. The inputs may include a surgical approach (e.g., medial parapetallar, subvastus, lateral). The inputs may include soft tissue management preferences, such as selective release of tight tissues, preservation of all soft tissues, aggressive release for tumor clearance, and selective release for growth preservation. The inputs may include a patellar management style/preference, including resurfacing and retention.

### Surgical Alignments

Mechanical alignment refers to prioritized restoration of the mechanical axis of the limb, typically as measured from the center of the hip to the center of the ankle, through the center of the knee. This approach prioritizes proper weight-bearing and joint loading of the limb or joint to prevent degenerative changes. It is commonly used in total knee arthroplasty (TKA) to ensure optimal prosthetic function.

Anatomical alignment refers to prioritized restoration of the patient's individual anatomical joint lines and soft tissue balance. This approach aims to preserve the natural kinematics and proprioception of the limb or joint. It is often used in knee reconstruction and osteotomy procedures to restore joint function and alleviate pain.

While the above examples may reflect certain surgeon styles, preferences, and/or workflows, user model 1210 may accommodate any type of surgical approach or methodology as input data. Programmable humanoid robot 120 may thus be configured based on surgeon styles, preferences, and/or workflows (e.g., a surgeon operating programmable humanoid robot 120).

### Workflows and Resection Techniques

Two primary surgical workflows are utilized in robot assisted total knee arthroplasty for pre-balancing the knee-the pre-resection workflow and the mid-resection workflow. The pre-resection workflow is typically used in cases where the deformity is correctable, and adjustments in the implant position may achieve both balance and targeted alignment. In this approach, the knee gap is balanced first, and then bone cuts are made sequentially without requiring major soft-tissue releases.

The mid-resection workflow is used when pre-balance cannot be achieved by adjusting the implant position alone. In such cases, the extension gap is balanced initially, followed by balancing the flexion gap. This approach is typically reserved for cases where there are more complex deformities or when the soft tissue is too tight to allow for pre-resection balancing. In such cases, two cuts initial cuts are made during the pre-resection workflow to provide access to the tibial side, which allows for the required soft-tissue releases to be performed.

### Surgical Arthrotomy Approach

In total knee arthroplasty (TKA), a user's choice of arthrotomy approach significantly influences surgical exposure, soft tissue preservation, and postoperative recovery. There are three commonly used methods for exposure: medial parapatellar, midvastus, and subvastus. The medial parapatellar approach is the most commonly used, and involves an incision along the medial border of the patella and patellar tendon. This method provides excellent visualization of the knee joint, which may facilitate precise component placement. However, this approach involves splitting the quadriceps tendon, which may impact early postoperative quadriceps function.

Alternatively, the midvastus approach involves an incision through the vastus medialis obliquus (VMO) muscle fibers, preserving the quadriceps tendon. This technique aims to maintain extensor mechanism integrity, which may be associated with quicker recovery and improved early postoperative quadriceps strength. However, this technique may offer limited exposure compared to the medial parapatellar approach, which may be challenging in patients with larger body habitus or complex knee anatomy.

Yet another option is the subvastus approach, in which the surgeon accesses the knee joint by lifting the vastus medialis muscle without cutting it. This method preserves both the quadriceps tendon and muscle, potentially resulting in less postoperative pain and faster rehabilitation. However, it is technically more demanding and may not provide sufficient exposure in all cases, particularly in patients with limited knee flexion or significant deformity.

### Patellar Management

In total knee arthroplasty (TKA), managing the patella involves two primary options: resurfacing and non-resurfacing. The surgeon's choice between resurfacing and non-resurfacing is influenced by factors like the patient's anatomy, the condition of the patellar cartilage, and surgeon preference.

Patellar resurfacing includes replacing the damaged cartilage on the underside of the patella with a prosthetic component, typically composed polyethylene. This approach aims to reduce anterior knee pain and improve joint function.

Non-resurfacing preserves the native patellar cartilage, relying on the existing anatomy to function with the new femoral component. This technique may avoid potential complications associated with resurfacing, such as patellar fracture or implant-related issues.

### Soft-Tissue Management

A collateral ligament release may be performed to address tightness in the medial or lateral collateral ligaments and may be performed for correcting varus or valgus deformities. Sequential release of these ligaments helps balance the knee joint thereby improving postoperative stability.

A posterior cruciate ligament (PCL) may alternatively be performed. In PCL-retaining TKA, preservation of the ligament may maintain natural knee kinematics. Alternatively, PCL-sacrificing or substituting designs may involve resecting the PCL and using a prosthesis with a cam-post mechanism to replicate its function. The decision of which soft-tissue management technique is used depends on patient-specific factors and surgeon preference.

Finally, a pie-crusting technique may be used for soft-tissue management. This method involves creating multiple small incisions in tight soft tissue structures, such as the iliotibial band or posterior capsule, to gradually release tension. This method is particularly useful for achieving balanced flexion and extension gaps without compromising ligament integrity.

The inputs to the surgical model 1212 may include, for example, data related to the specific techniques and movements of a human surgeon during a medical procedure. For example, a specific movement of one or more body parts of a surgeon may be mapped to a specific portion of a surgical procedure. In this example, a certain cut performed by a surgeon may be mapped to one or more body and/or limb (including fingers and hands) movements. It should be understood that throughout this disclosure that any reference to a “surgeon” may be interchangeably used with the term “user”, unless otherwise noted. Inputs to surgical model 1212 may include motion capture data of a human surgeon and associated medical instruments, including position, orientation, and velocity/acceleration via systems such as motion capture, trackable instruments (e.g., accelerometers, gyroscopes), and/or robotic telemetry data (e.g., based on telemanipulated robot data). Other systems that may be employed for user motion capture may include augmented reality headsets, virtual reality headsets, mixed reality headsets, infrared systems, and the like. Surgical model 1212 may be at least partially based on one or more of a list of parameters associated with a given portion of a surgical procedure.

Inputs to surgical model 1212 may include force and/or haptic feedback data associated with how a surgeon moves and/or applies force(s) during a medical procedure (e.g., gripping, cutting, etc.). Such data may be generated by one or more force and/or torque sensors operably coupled to one or more medical instruments and one or more tactile sensors operably coupled to one or more medical instruments. Inputs to surgical model 1212 may include visual media (e.g., image and video) data, which may be provided by one or more surgical cameras (e.g., an endoscopic camera), one or more external cameras (e.g., an operating room camera and/or a camera externally mounted to a surgeon or other human within the medical procedure room). Hand data associated with the surgeon may also be utilized, including wearable glove-based sensors that track finger movements, joint angles, inadvertent movements such as hand tremors, and/or grip strength, as well as hand data generated by wearable inertial measurement units (IMUs) to track the surgeon's hand movements in three dimensional space. In an example, a user-worn headset, such as an augmented reality headset, may capture a user's hand and finger movements. Additional data may be generated by various means, including hand size and dexterity, posture, height, and physical stamina (e.g., compensation for surgeon fatigue). Improvements to training models may generally be based on data generated during humanoid robot surgeries. The vision and/or camera system outputs may be fed into a machine learning model which may output tracking information in a similar manner to the aforementioned IMUs and glove trackers.. Adjustment of surgical model 1212 (and/or surgical phenotype selection model 121) may utilize any of the aforementioned data.

Audio data associated with the medical procedure may be captured and used an input for surgical model 1212. Audio data may be generated simultaneously with video data, or may be generated independently by one or more recording devices (e.g., microphones). Audio captured may include instructions, comments, and/or commands issued by one or more medical professionals during a medical procedure. Spatial audio may be captured from microphone areas to provide depth and/or location data to various audio sources within the operating room. Audio may also be used to capture specific medical information. For example, audio associated with a bone saw may be indicative of bone quality and/or the complexity of a given case and itself may be used as surgical feedback during remote surgical applications.

One or more biological metrics may be captured and input to surgical model 1212. For example, heart rate monitors, blood pressure sensors, and/or pupil dilation may be captured during a medical procedure. These data points may be associated with periods of heightened focus during a medical procedure, for example. Such data may be clinically valuable for programmable humanoid robot 120 to indicate when surgical motions of the robot should be slowed, processing time increased, etc.

Medical procedure workflows may be input to surgical model 1212. For example, clinical decision making and clinically relevant tasks such as when and/or where a certain cut is to be made may be captured on video, and may be labeled by AI systems 130 and/or a human operator. A labeled sequence of surgical steps may thus be used as input data for surgical model 1212. In some aspects, one or more sensored implants may provide input data to surgical model 1212.

The outputs from surgical model 1212 may include clinically relevant motions and tasks to be performed by the humanoid robot, including, for example, how much force and torque to apply to the relevant joints or limbs in the completion of those tasks. Furthermore, the outputs from surgical model 1212 may be inputs into surgical recommendations based on the feedback from the simulated activities to, for example, identify the most optimal means of performing a given surgical action.

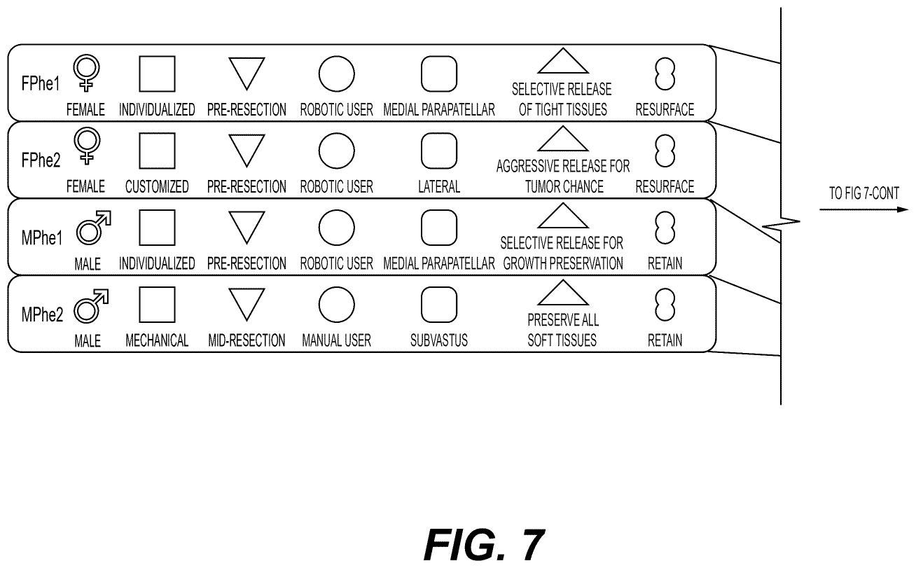

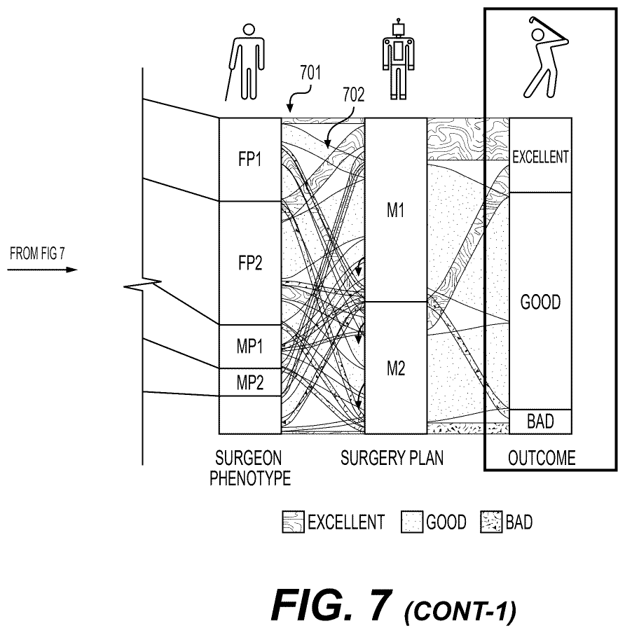

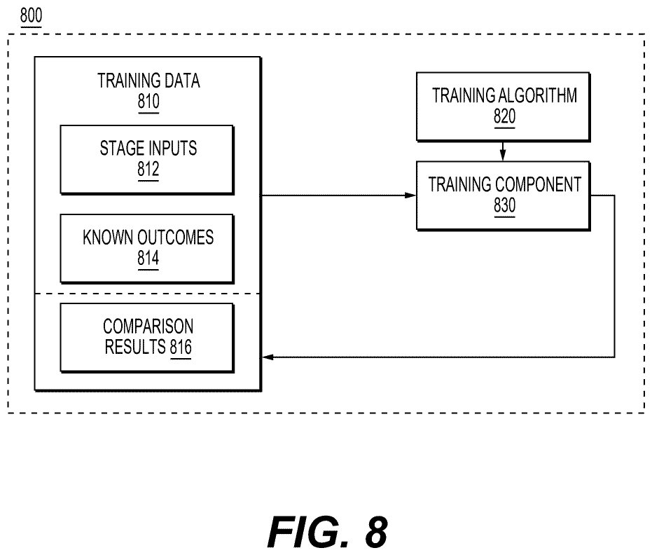

The inputs, outputs, and training of user model 1210 and surgical model 1212 will be discussed in more detail below with regard to FIGS. 7-8. User model 1210 and surgical model 1212 may be continuously learning models as well, where outputs from the models may be used as inputs for future iterations of the model. A machine learning model is generally trained using training data, e.g., experiential data or samples of input data, which are fed into the model in order to establish, tune, or modify one or more aspects of the model, e.g., the weights, biases, criteria for forming classifications or clusters, or the like. The training data may be generated, received, or otherwise obtained from internal or external resources. Aspects of a machine learning system may operate on an input linearly, in parallel, via a network (e.g., a neural network), or via any suitable configuration. By virtue of such training, a machine learning model is converted from an un-trained and un-specific model to a model that is unique to and specifically configured for the particular purpose for which it is trained. In an example, training of a machine learning model is analogous to a method of production in which the article produced is the trained model having unique characteristics by virtue of its particular training. Moreover, the result of training a machine learning model using particular training data and for a particular purpose results in a technical solution to an inherently technical problem.

The execution of the machine learning model may include deployment of one or more machine learning techniques, such as linear regression, logistical regression, random forest, gradient boosted machine (GBM), deep learning, or a deep neural network. Supervised or unsupervised training may be employed. For example, supervised learning may include providing training data and labels corresponding to the training data, e.g., as ground truth. One sensor can serve as ground truth for different machine learning models. In an example actuator and/or encoder values may serve as ground truth for training visual systems. Unsupervised approaches may include clustering, classification, or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc. Alternatively, reinforcement learning may be employed for training. For example, reinforcement learning may include training an agent interacting with an environment to make a decision based on the current state of the environment, receive feedback (e.g., a positive or negative reward based on accuracy of decision), adjusts its decision to maximize the reward, and repeat again until a loss function is optimized. In some aspects, imitation learning may be used to train the model to perform tasks by observing and mimicking demonstration of one or more actions or procedures. In some aspects, the systems, devices, and methods of the disclosure may employ end-to-end AI, which may be designed to learn and perform a task directly from raw inputs to final outputs, without relying on intermediate manual feature extraction or separate processing stages.

As discussed above, the humanoid robot may include one or more sensors 123, which may include, for example, IMUs, strain gauges, temperature sensors, gyroscopes, accelerometers, etc. An IMU may include, for example, three gyroscopes and three accelerometers, where a first, second, and third gyroscope and a first, second, and third accelerometer are respectively aligned to three perpendicular axes. Each gyroscope may measure an angular velocity corresponding to a rotation about an axis. In other examples, the IMU may include any number of gyroscopes and any number of accelerometers, may only include one or more gyroscopes and not include accelerometers, or may only include one or more accelerometers and not include gyroscopes.

Each accelerometer may measure a change in motion (acceleration) corresponding to one of the axes. The IMU may include up to nine degrees of freedom (DOF), which may include accelerations, gyroscopic velocities, and magnetometer values for 3-dimensional space. For example, the IMU may include up to 9-DOF, 6-DOF, or 3-DOF, and is not limited to the above-described arrangement. IMU 220 is configured to measure 6 degrees of freedom comprising translation movement along the X axis, Y axis, and Z axis as well as rotational movement such as yaw, roll, and pitch around each axis. Sensors 232 can be added to measure one or more parameters of interest and may differ depending on the application of the device.

The IMU may include one or more micro-electro mechanical (MEMs) integrated circuits. For example, one or more of the gyroscopes or accelerometers may be or include a MEMs integrated circuit. A form factor of a MEMs gyroscope integrated circuit or MEMs accelerometer integrated circuit may support placement in a prosthetic component or coupling to a prosthetic component or bone surface to measure alignment of the muscular-skeletal system. The MEMs gyroscope may have a resonating mass that shifts with angular velocity and output a signal corresponding to (e.g., proportional to) the angular velocity of the IMU. A MEMs accelerometer may have a mass-spring system that shifts in response to an exerted acceleration, e.g., counter to a bias of a spring in the mass-spring system.

Sensors 123 may include other sensors, such as strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, etc. Measurement data from the IMU and/or other sensors may be transmitted to a computer system 126 or to AI systems 130 to process and/or display forces, alignment, range of motion, and/or other information from sensors 123. For example, measurement data from the IMU and/or other sensors may be transmitted wirelessly to a computer or other electronic device to be processed (e.g. via one or more algorithms) and displayed on an electronic display.

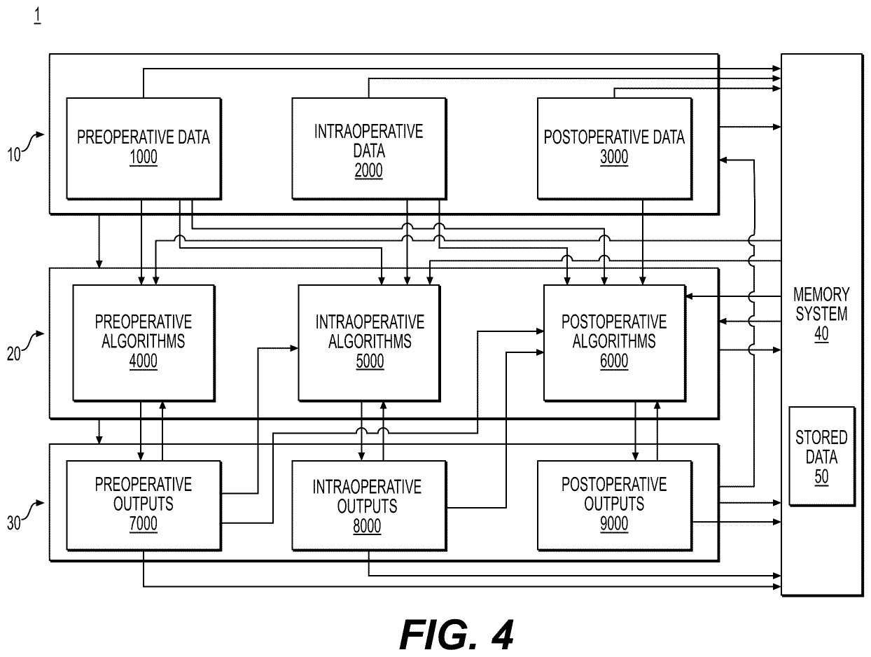

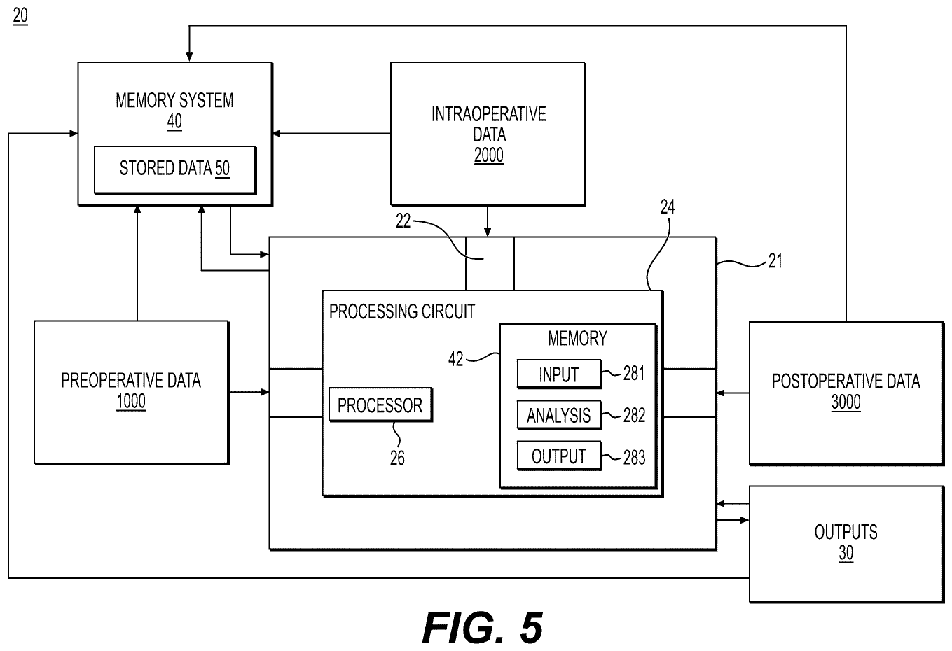

Different strains, loads, pressures, forces, etc. measured by each strain gauge sensor and may be processed to determine a load magnitude and location of the load applied. The measured strains and/or other data may be transmitted to the system 20 described with reference to FIGS. 4 and 5, or another computing platform to calculate load parameters, such as magnitude, location, direction, etc. of an applied load, force, etc. of a joint (e.g., hip joint) in real time, which may then be visualized on a display. Sensors 123 may be positioned anywhere on or in the humanoid robot 120. In some examples, external sensors may be used for testing with the humanoid robot 120, such as image sensors or other types of sensors external to the humanoid robot 120.

One or more imaging devices 124 may also be positioned on the humanoid robot 120 to provide computer vision as an input to the computer systems 126 for control of the humanoid robot 120 and as inputs to user model 1210 and surgical model 1212 in the operation of tasks by the humanoid robot 120. For example, the computer vision provided by the imaging devices 124 may be used for object detection or obstacle detection when performing a given task. The computer vision may also aid in dynamic and adaptive testing, where humanoid robots 120 can perform dynamic movements and adapt to changing conditions, more accurately replicating human behavior and providing more realistic testing conditions. Humanoid robot 120 may include or more displays (e.g., monitors) on various portions of the humanoid robot 120 (e.g., the chest) to replace or compliment the display of an external navigation device to aid with required visualization by users in the operating room.

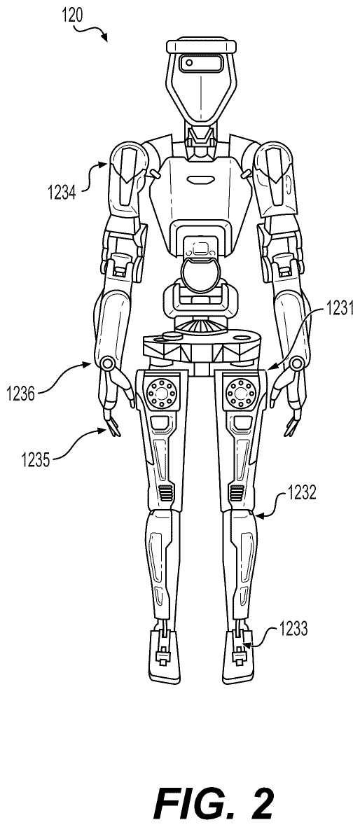

FIG. 2 is an exploded view of a humanoid robot 120 with a plurality of mechanical limbs, joints, and/or appendages (henceforth “electromechanical systems 125”), according to aspects of this disclosure. Advantageously, humanoid robot 120 may be configured to control multiple electromechanical systems 125 simultaneously. Current telemanipulated robots may lack sufficient dexterity, flexibility, and/or adaptability to various working environments and/or medical procedure types. Implementation of a humanoid with mechanical controls for either hip 1231, knee 1232, ankle 1233, shoulder 1234, fingers 1235, and/or wrist 1236 would provide a novel method for performing various medical procedures based on the movements and styles of users in clinically relevant environments. Generated field data could then be used to improve future control methods or develop a predictive algorithm for an expected outcome of a procedure based on data generated from other patients. The electromechanical systems 125 may be operably coupled to humanoid robot 120, with an example of a coupling discussed below. The humanoid robot 120 may collect information related to electromechanical systems 125 when performing a medical procedure. It will be appreciated that while the above mentioned hip 1231, knee 1232, ankle 1233, shoulder 1234, fingers 1235, and/or wrist 1236 are labeled and described in FIG. 2, one of ordinary skill in the art will appreciate that various other portions of humanoid robot 120 may include electromechanical systems 125 (e.g., neck, abdominal section, elbow, etc.) that may all generate data similarly to hip 1231, knee 1232, ankle 1233, shoulder 1234, fingers 1235, and/or wrist 1236. Based on modification of surgical phenotype model 121 (including user model 1210 and/or surgical model 1212), various parameters of electromechanical systems 125 may be adjusted. For example, actuator torque, actuator power, range of motion of various electrometrical limbs and joints, speed of various components and limbs (including a default movement speed) may be adjusted. Additional parameters of humanoid robot 120 may be adjusted, including kinematic parameters such as degrees of freedom of various electromechanical joints and/or limbs, electromechanical joint stiffness, center of mass, gait, stability, and any other suitable biomechanical property or parameter of a human user to be applied to humanoid robot 120 and/or electromechanical systems 125. It will be understood that any electromechanical limb or appendage may be controlled or manipulated by a user. For example, a surgeon may move a particular finger and the humanoid robot 120 may move its finger(s) 1235 in a similar manner to replicate the movement. The various movements of a user may thus be mapped 1:1 to the various electromechanical limbs, appendages, and/or systems of humanoid robot 120.

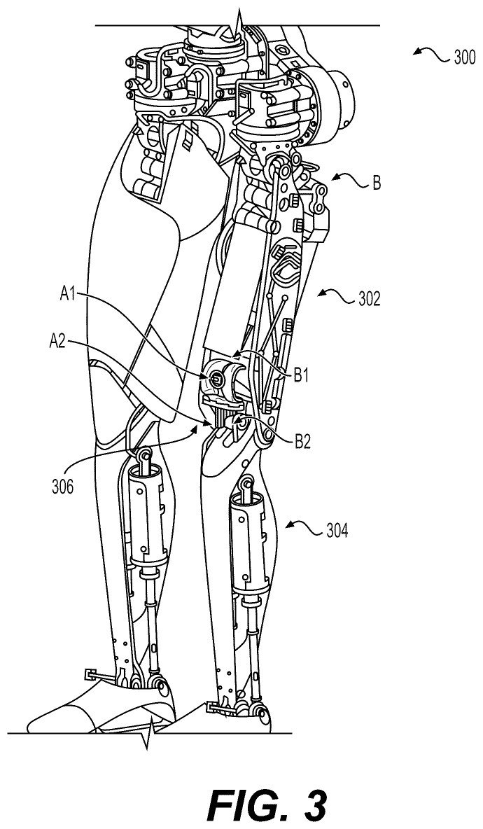

FIG. 3 is an illustration of a lower portion of a humanoid robot 120. In particular, FIG. 3 depicts a robotic leg 300 of a humanoid robot 120 with a knee 306 disposed between an electromechanical upper leg system 302 and an electromechanical lower leg system 304. The electromechanical systems 125 such as the upper leg system 302 and the lower leg system 304 may include actuators, motors (e.g., servo, linear, DC), hydraulic and/or pneumatic systems, linkages, etc. that may be manipulated or designed to mimic or simulate bone, muscle, or other soft tissues, such as, for example, tendons, ligaments, etc., that may be tuned by the surgical phenotype selection module 121 to simulate specific surgical phenotypes. Surgical phenotypes will be discussed in more detail with reference to FIG. 7 below.

FIG. 4 illustrates an electronic data processing system 1 for collecting, storing, processing, and outputting data throughout the course of treatment of a patient, and may be incorporated into any of the measurement systems discussed herein.

Referring to FIG. 4, input information 10 may be input into a system or module 20 to generate output information 30, which may be fed back into system 20 as input information 10. System 20 may be an artificial intelligence (AI) and/or machine learning system. System 20 may include an AI module of AI system 130 (shown in FIG. 1), which may include or communicate with a memory system 40 configured to store the plurality of inputs or input information 10, outputs or output information 30, and stored data 50 from prior patients and/or prior procedures. The input information 10 and output information 30 of an instant procedure may also become stored data 50 and/or used as input information 10 into system 20 and/or memory system 40. Although certain information is described in this specification as being input information 10 or output information 30, due to the continuous feedback loops of data (which may be anchored by memory system 40), the input information 10 described herein may alternatively be determinations or output information 30, and the output information 30 described herein may also be used as input information 10. For example, some input information 10 may be directly sensed or otherwise received, and other input information 10 may be determined or output based on other input information 10.

The input information 10 may include preoperative data 1000, intraoperative data 2000, and post-operative data 3000. System 20 may perform a plurality of algorithms, such as preoperative algorithms 4000, intraoperative algorithms 5000, and postoperative algorithms 6000 to generate the output information 30. The output information 30 may include preoperative outputs 7000, intraoperative outputs 8000, and postoperative outputs 9000. Some or all of the preoperative outputs 7000, intraoperative outputs 8000, and postoperative outputs 9000 may include determinations such as guidance for medical procedures, guidance for pre-operative or pre-habilitation treatment plans, guidance for post-operative or recovery plans, etc., as will be described in more detail hereinafter. System 20 may include one or more algorithms or modules configured to aggregate results from multiple preoperative algorithms 4000, intraoperative algorithms 5000, and/or postoperative algorithms 6000 to compile algorithm determinations for certain outputs (e.g., surgical plans, medical treatment plans, or instructions). As shown by the arrows in FIG. 4, the preoperative outputs 7000, intraoperative outputs 8000, and postoperative outputs 9000 may become inputs into system 10 and/or memory system 40.

Preoperative data 1000 may be data collected, received, and/or stored prior to an initiation of a medical treatment plan or medical procedure. Intraoperative data 2000 may be data collected, received, and/or stored during a medical procedure, for example using the programmable humanoid robot 120. Postoperative data 3000 may be data collected, received, and/or stored after completion of the medical treatment or medical procedure.

FIG. 5 illustrates an exemplary system architecture for system 20. Referring to FIG. 5, an AI module 21 may be implemented using one or more computing platforms. Examples of one or more computing platforms may include, but are not limited to, smartphones, wearable devices, tablets, laptop computers, desktop computers, Internet of Things (IoT) device, remote server/cloud based computing devices, or other mobile or stationary devices. The AI module 21 may also include one or more hosts or servers connected to a networked environment through wireless or wired connections. Remote platforms may be implemented in or function as base stations, which may also be referred to as Node Bs including but not limited to, evolved Node Bs (eNBs) and next-generation node Bs (gNBs) as well as future contemplated networking standards such as 6G). Remote platforms may also include web servers, mail servers, application servers, etc.

The AI module 21 may include at least one communication module or interface 22 and a processing circuit 24. The processing circuit 24 may include one or more processors 26 and a memory or storage 42. The memory or storage 42 may be a part of the memory system 40. The memory system 40 is shown in FIG. 5 as providing separate storage from the AI module 21 to exemplify that large amounts of data (e.g., stored data 50) may be stored separately and sent to the AI module 21 via communication modules 22 when needed or where appropriate. However, the memory system 40 may be a part of a computing platform for the AI module 21.

The AI module 21 may be configured to receive the plurality of inputs 10 (the preoperative data 1000, intraoperative data 2000, and post-operative data 3000), and/or stored data 50 from prior procedures or patients, via the communication module 22. The preoperative data 1000, intraoperative data 2000, and post-operative data 3000 may be received via manual input or from various sensors. The plurality of inputs 10 may be stored in memory 42 and/or memory system 40. The plurality of input information 10 may be analyzed by processor 24 to determine patterns, such as but not limited to, patterns of movement, wear, force, or displacement. The AI module 21 may be configured to perform the preoperative algorithms 4000, intraoperative algorithms 5000, and postoperative algorithms 6000 via the processing circuit 24, and to generate the output information 30 via the processor 26.

The communication module 22 may enable wireless communications between the system 20, including among a network of humanoid robots 120, and the various sensors or data collection devices described herein. Humanoid robtos 120 may be connected across a network to access shared information, such as internal user data and external sensor data acquired from additional networked humanoid robots 120. The communication module 22 may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, 6G, Bluetooth, near field communication (NFC), radio frequency identifier (RFID), ultrawideband (UWB), satellite internet systems, etc.). The communication module 22 may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink). The communication module 22 may include a Bluetooth module, Wi-Fi module, etc. to receive the input information 10. For example, communication module 22 may include an Ethernet card and port for sending and receiving data via an Ethernet-based communications link or network. In another example, communication module 22 may include a Wi-Fi transceiver for communication via a wireless communications network.

The processing circuit 24 may be configured to implement various functions (e.g., calculations, processes, analyses) described herein. The processor 26 may be implemented as a general purpose processor or computer that may be on-board or in the cloud (e.g. via a remote system, etc.), special purpose computer or processor, microprocessor, digital signal processor (DSPs), an application specific integrated circuit (ASIC), one or more field programmable gate arrays (FPGAs), a group of processing components, one or more processors based on a multi-core processor architecture, GPGPUs (General Purpose Graphical Processing Units), one or more quantum processors, or other suitable electronic processing components. The processor 26 may be configured to perform machine readable instructions, which may include one or more modules implemented as one or more functional logic, hardware logic, electronic circuitry, software modules, etc. In some cases, the processor 26 may be remote from one or more of the computing platforms comprising the module 21 and/or system 20. The processor 26 may be configured to perform one or more functions associated with the AI module 21, such as precoding of antenna gain/phase parameters, encoding and decoding of individual bits forming a communication message, formatting of information, and overall control of one or more computing platforms comprising the AI module 21, including processes related to management of communication resources and/or the communication module 22.

The memory 42 may provide an example of the types of devices comprising the memory system 40. The memory 42 may be one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory 42 may be or include volatile memory or non-volatile memory (e.g., semiconductor-based memory device, a magnetic memory device and system, an optical memory device and system, fixed memory, or removable memory). The memory 42 may include database components, object code components, script components, or any other type of information structure to support the various activities described herein. In some aspects or embodiments, the memory 42 may be communicably connected to the processor 26 and may include computer code to execute one or more processes described herein. The memory 42 may contain a variety of modules, each capable of storing data and/or computer code related to specific types of functions. In some embodiments, the memory 42 may contain several modules related to medical procedures, such as an input module 281, an analysis module 282, and an output module 283. The input module 281 may receive input information 10, and the output module 283 may output (e.g., display or transmit) output information 30. The analysis module 282 may include and/or operate the preoperative algorithms 4000, intraoperative algorithms 5000, and postoperative algorithms 6000. In some aspects, a machine learning model may be used to filter data and to only store data that is novel or anomalous to reduce memory and/or storage use. The AI module 21 and/or system 20 need not be contained in a single housing. Rather, components of the AI module 21 may be located in various different locations or even in a remote location (e.g. remote cloud server system, etc.). Components of the module 21, including components of the processor 26 and the memory 42, may be located, for example, in components of different computers, robotic systems, devices, etc. used in surgical procedures.

The pre-operative data 1000, intraoperative data 2000, and post-operative data 3000 may be collected using preoperative, intraoperative, and postoperative measurement systems. The intraoperative measurement system may include, for example, the programmable humanoid robot 120 and elements of the environment 100 described with reference to FIG. 1. The preoperative algorithms 4000, intraoperative algorithms 5000, and postoperative algorithms 6000 may be used to generate preoperative outputs 7000, intraoperative outputs 8000, and postoperative outputs 9000.

Preoperative data 1000 may include any information collected by memory system 40 prior to a medical procedure, such as a surgical procedure or other patient treatment event. The preoperative data 1000 may include information on demographics, lifestyle, medical history, electromyography (EMG), planned procedure, psychosocial information, bone imaging, soft tissue imaging, bone density, biometrics, and kinematics. This list, however, is not exhaustive and preoperative data 1000 may include other patient specific information. Some of the preoperative data 1000 may be directly sensed via one or more devices, may be manually entered by a medical professional, patient, or other party, and other preoperative data 1000 may be determined (e.g., using a preoperative algorithm 4000) based on directly sensed information, input information, and/or stored information from prior medical procedures.

Demographics may include patient age, gender, height, weight, nationality, body mass index (BMI), etc. Lifestyle may include information on smoking habits, exercise habits, drinking habits, eating habits, fitness, thrill-seeking habits and/or risk adverse traits, a type of vehicle a patient drives and movements associated with entering and exiting the vehicle, a type of house or residence the patient lives in and movements associated with climbing and descending stairs, bending movements during daily activities, etc.

Medical history may include allergies, disease progressions, addictions, prior medication use, prior drug use, prior infections, comorbidities, prior surgeries or treatment, prior injuries, prior pregnancies, utilization of orthotics, braces, prosthetics, or other medical devices, etc. EMG information may include information on a muscle response or electrical activity in response to a nerve's stimulation. A patient's medical history may include visit notes and charts, which humanoid robot 120 can gather by recording conversations between the patient and staff during a visit. Humanoid robot 120 may then generate summaries, charts, or notes and store this data in the patient's electronic records.

Information on a planned procedure may include information about a planned site of the procedure, a disease or infection state, type of procedure to be performed, etc. Alternatively or in addition thereto, a planned procedure may include a surgeon's surgical or other procedure or treatment plan (planned steps or instructions on incisions, bone cuts, implant design, implant alignment, etc.) that was manually prepared by a surgeon and/or previously prepared using one or more algorithms. Psychosocial information may include perceived pain, stress level, anxiety level, mental health status, other feelings and psychosocial data, and other patient reported outcome measures (PROMS). Psychosocial information may include mental health status and/or information from a Veteran's Rand-12 (VR-12) mental component summary (MCS).

Bone imaging data may include morphology and/or anthropometrics 1082 (e.g., physical dimensions of internal organs, bones, etc.), fractures, slope or angular data, tibial slope, posterior tibial slope or PTS, bone density 1090 (e.g., bone mineral or bone marrow density, bone softness or hardness, or bone impact), etc. Bone density 1090 may be collected separately from bone imaging information 1080 and/or may be collected using, for example, using indent tests or a microindentation tool. Bone imaging data 1080 may be converted into various 3D computer-aided-design (CAD) formats, which may be used in enhanced planning algorithms during the pre-operative stages. Bone imaging data 1080 may not be limited to strictly “bone” and may be inclusive of other internal imaging data, such as of cartilage, soft tissue, or ligaments.

Bone imaging data may include or be used to determine alignment data. Bone imaging data, alignment data, and/or morphology and/or anthropometrics may include data on bone landmarks (e.g., condyle surface, head or epiphysis, neck or metaphysis, body or diaphysis, articular surface, epicondyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e.g., lengths or thicknesses of a tibia or femur). Bone imaging data, alignment data, and/or morphology and/or anthropometrics may also include data on soft tissues for ligament insertions and/or be used to determine ligament insertion sites. For example, bone density may be determined from bone imaging data and may be used to locate or determine a ligament insertion site to balance a knee, or MRI or fMRI data could give additional soft tissue information. Similarly to CAD digitization of bone imaging data, soft tissue insertion points may also be generated as 3D CAD files and used as an input for a soft-tissue model within one or more AI systems or used as a surgical planning input parameter.

Bone imaging data, alignment data, and/or morphology and/or anthropometrics may include lower extremity mechanical alignment, lower extremity anatomical alignment, femoral articular surface angle, tibial articular surface angle, mechanical axis alignment strategy, anatomical alignment strategy, natural knee alignment strategy, femoral bowing, tibial bowing, patello-femoral alignment, coronal plane deformity, coronal plane deformity that can be passively correctable, sagittal plane deformity, extension motion, flexion motion, anterior cruciate ligament (ACL) ligament intact, posterior cruciate ligament (PCL) ligament intact, knee motion in all three planes during active and passive range of motion in a joint, three dimensional size, proportions and relationships of joint anatomy in both static and motion, height of a joint line, lateral epicondyle, medial epicondyle, lateral femoral metaphyseal flare, medial femoral metaphyseal flare, proximal tibio-fibular joint, tibial tubercle, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance.

Biometric data may include resting heart rate or heat rate variability, electrocardiogram data, breathing rate, temperature (e.g., internal or skin temperature), skin moisture, oxygenation, sleep patterns (e.g., heart rate variability or HRV, REM cycle data, type of sleep such as REM, deep, or light, sleep frequency, time asleep versus time awake, disturbances in the sleep or periods of movement, patterns in sleep timing or time of day asleep, etc.), and/or activity frequency and intensity. Biometrics may include patient-specific or unique characteristics, such as fingerprint data, DNA or RNA signatures, etc.

Kinematic data may include movement or position information at various anatomical areas or locations, muscle function or capability, and range of motion data. Additional kinematics data may include strength measurements and/or force measurements. For example, kinematic data may include data used to determine a push-off power, force, or acceleration, or a power, force, or acceleration at a toe during walking. Range of motion data may include a range of motion at one or more joints, such as an angular range or axes of joint motion, or rotation, flexion or extension data. For example, kinematic data may include a flexion value, where a flexion value of 180 degrees ±3 degrees may indicate a full extension of a joint, and any value other than 180 degrees ±3 degrees may indicate a joint in flexion where bones on either side of the joint intersect to form an angle other than 180 degrees. Kinematic data may include dynamic information, speed or acceleration information, torque or force information, etc. Some of this information may be estimated or determined based on raw data from motion sensor systems and/or other sensors. For example, kinematic data may include how quickly a patient can bend a joint, sit down, stand up, a push-off power during walking, etc. Kinematics may also include steps (e.g., measured by a pedometer) and/or measured gait, including gait metrics such as stride length, stance, cadence, etc. Kinematic data may include a number of fall events and/or disoriented events (e.g., measured by an accelerometer, mobile device, etc.)

Kinematic data may include swaying or other movement which would indicate an unsteady balance of a patient, such as postural sway at the hips, knees, or neck. Kinematics may include pendulum knee drop information. Kinematic data may also include and/or indicate frailty, fall risk, and/or joint stiffness (e.g., based on a speed or ease of how a joint is moved through a range of motion).

The kinematic data may include measurements in relation to a leg axis system, such as alignment data or other anatomical measures. Alignment data may be obtained using kinematics information and/or range of motion information, bone imaging data and/or morphology/anthropometrics data, etc. In this way, alignment data may also be a type of preoperative output 7000. Anatomical measures and/or alignment data may include arithmetic hip-knee-ankle angle or aHKA, anatomical hip-knee-ankle angle, medial proximal tibial angle or MPTA, lateral distal femoral angle or LDFA, mechanical axis alignment, anatomic alignment, natural knee alignment, gap balancing, measured resection, etc., and these values may be combined. For instance, a joint line may be a sum of MPTA and LDFA, and a hip-knee-ankle angle or HKA may be a difference between MPTA and LDFA. These values may be used as coordinates on a 2D plane to describe a patient's knee anatomy and/or combined with additional multiplanar data to describe patient knee anatomy in 3D space.

Preoperative data 1000 and/or information stored in the memory system 40 may also include known data and/or data from third parties, such as data from the Knee Society Clinical Rating System (KSS) or data from the Western Ontario and McMaster Universities Osteoarthritis Index (WOMAC).

System 20 may collect pre-operative data 1000 from electronic devices storing electronic medical records (EMR), patient/user interfaces or applications such as tablets, computers, and cellular phones, diagnostic imaging systems, mobile devices, a motion sensor, pressure sensor, and/or kinesthetic sensing systems, and electromyography or EMG systems. The devices of the preoperative measurement system may each include one or more communication modules (e.g., Wi-Fi modules, Bluetooth modules, etc.) configured to transmit preoperative data 1000 to the memory system 40, system 20, to each other, etc. System 20 may use other types of stimulation systems (e.g., configured for a kinematic or EMG response) to collect preoperative data 1000.

System 20 may collect patient reported data, practitioner assessments, etc., using EMR. For example, EMR may be used to collect data on demographics, medical history, biometrics, and information about a planned procedure. Patient and/or user interfaces may be implemented on mobile applications and/or patient management websites or interfaces. Patient interfaces may present questionnaires, surveys, or other prompts for patients to enter psychosocial information 1060 such as perceived or evaluated pain, stress level, anxiety level, feelings, and other patient reported outcome measures (PROMS). Practitioners may also report psychosocial information (e.g., qualitative assessments or evaluations) via patient interfaces or other interfaces. Patients may also report lifestyle information via patient interfaces. These patient interfaces may be executed on other devices disclosed herein (e.g., using mobile devices or other computers).

System 20 may collect imaging information from diagnostic imaging systems, which may include computed tomography (CT) scans, magnetic resonance imaging (MRI), x-rays, radiography, ultrasound, thermography, tactile imaging, elastography, nuclear medicine functional imaging, positron emission tomography (PET), single-photon emission computer tomography (SPECT), etc. System 20 may use these diagnostic imaging systems to collect bone imaging information, including morphology and/or anthropometrics fractures, and bone density (e.g., bone mineral density or bone marrow density).

Mobile devices may include smartwatches, smartphones, tablets, augmented reality headsets, virtual reality headsets, mixed reality headsets, and other devices known in the art. Mobile devices may execute patient interfaces. In some examples, mobile devices may include sensors and/or applications, which system 20 may use to collect biometrics and other types of patient specific data. For example, mobile devices may use cameras, light sensors, barometers, global positioning systems (GPS), accelerometers, temperature sensors (e.g., battery temperature sensors), and/or pressure sensors. In some examples, mobile devices may measure heart rate, electrocardiogram data, breathing rate, temperature, oxygenation, sleep patterns, and also activity frequency and intensity.

System 20 may use EMG systems to collect EMG data. EMG systems may include one or more electrode attached to skin or muscle to measure electrical activity and/or responses to nerve stimulation. System 20 may use EMG data to determine nerve damage or disease information. EMG data may include information on muscle activity of various body segments including knee, hip, ankle, tibialis anterior, foot, lower back, shoulder, wrist, elbow, forearm, neck, etc.

System 20 may use motion sensor and/or kinesthetic sensing systems, which may include motion capture (mocap) systems, external motion sensors, wearable sensors, and/or sensors machine vision (MV) technology. Motion sensor systems may measure motion using an optical or light sensor, an accelerometer, a gyroscope, a magnetometer, a compass, barometer, a global positioning system (GPS), a pressure sensor, etc.

System 20 may use motion capture systems, which may include markerless motion capture systems and other motion sensors (e.g., wearable sensors) to collect kinematics and range of motion data. External motion sensors may include cameras, optical sensors, infrared sensors, ultrasonic sensors, etc. mounted, for example, in an operating room to monitor motion, heat, etc. Motion capture may also utilize headsets (AR/MR/VR) and/or mounted motion capture systems.

Preoperative outputs 7000 may be determined via one or more preoperative algorithms 4000. The preoperative algorithms 4000 may also consider and/or analyze other previously stored data 50 of memory system 40 to determine preoperative outputs 7000. Preoperative outputs 7000 may include a prehabilitation plan, a procedure, medical treatment, or surgical plan, a postoperative plan, a bone density score, a fall risk or stability score, a morphology score, an EMG score, an activity quality score, a joint stiffness score, a patient readiness score, psychosocial score, a b-score or bone shape score, a push-off power score, and a fracture risk score. This list is not exhaustive, however. A “treatment course” or “course of treatment” may refer to any one of or all of the prehabilitation plan, procedure plan, and postoperative plan and/or their intraoperatively determined and postoperatively determined analogs described later.

The procedure, medical treatment, or surgical plan may include instructions for a surgeon in preparing for and/or performing a procedure (e.g., surgery) on the patient. For example, when the procedure plan is a surgical plan for installation of an implant, the surgical plan may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000), a list of steps for the surgeon to perform, a list of tools that may be used, a planned operating room layout (e.g., positions and/or movement of objects or people in the operating room, such as staff, surgeons, medical or surgical robot, operating room table, patient, cameras, GUI, sensors, or other equipment), etc. The procedure plan may also include predictive or target outcomes and/or parameters, such as target postoperative range of motion and alignment parameters, target fall risk or fracture scores, activity quality scores, and joint stiffness scores. These target parameters may be compared postoperatively to corresponding measured postoperative data 3000 and/or determined postoperative outputs 9000 to determine whether an optimized outcome for a patient was achieved. Furthermore, surgical planning information may be used to achieve a specific phenotype plan where the data is used in a closed feedback loop to inform surgical decisions or assess implants iteratively based on different surgical decisions, re-evaluating device performance with alternative configurations, and/or monitoring the performance of a continued or new prostheses device.

A design of the implant may include, for example, curvatures, shapes, or thicknesses and/or shimming parameters corresponding to a patient's anatomy (e.g., from bone imaging data). For example, a design of the implant and/or prosthetic may be configured to match an arc of curvature of the implant with an arc of curvature of the native femoral condyle of the patient, an arc or curvature of a socket area or acetabulum, an arc or curvature of a glenoid or humerus, an arc or curvature of a tibial condyle, etc. Aspects disclosed herein may be applied to a custom knee implant design, custom hip implant design, custom partial knee or hip implant design, or custom design of any other implant design for any other part of a patient's anatomy. The design of the implant may also include materials of the implant and/or placement of implants of autologous tissue, allograft tissue, and/or synthetic materials. The design of the implant may include thicknesses, a number of shims configured to be stacked and/or removed, a size of an added shim, or other dimensions configured to adjust a fit or tightness of the implant.

The postoperative plan may include plans similar to the prehabilitation plan such as an exercise program configured to decrease a recovery time of the patient. The postoperative plan may further include a medication plan (e.g., pain medication plan including a type, dosage, and/or tapering of pain medication) and/or a discharge plan including a length of stay in a hospital. The postoperative plan may include a schedule of follow-up visits with a practitioner, surgeon, physical therapist, etc. The postoperative plan may also include a plan for revision surgeries or future additional surgeries, though the procedure plan may be configured to reduce a likelihood of revision procedures or surgeries. Like the prehabilitation plan and procedure plan, the postoperative plan may be based on other preoperative outputs 7000. For example, the postoperative plan may include an exercise program configured to target muscles based on the patient's lifestyle (e.g., frequency of climbing stairs or frequency of entering/exiting cars), the fall risk score, and/or the fracture score. The procedure plan may be updated and/or modified based on intraoperative information 2000 and postoperative information 3000.

The bone density score may be calculated from bone density data, bone imaging data (e.g., morphology/anthropometrics data), medical history, and/or other information input by a patient or practitioner. The bone density score may be implemented as a T-score, defined as the difference between a patient's bone mineral density and 0, which is the bone mineral density of a healthy young adult, where a higher score correlates to a greater bone density, but aspects disclosed herein are not limited.

The fall risk score may be calculated from kinematics, range of motion (e.g., postural sway), and alignment. The fall risk score may be paired with or be calculated based on lifestyle data. For example, the fall risk score may be calculated on a mobile device, be updated based on information sensed by the mobile device, and be displayed on the mobile device (e.g., in a fall risk tracking app). The fall risk score may also be based on other preoperative outputs and/or qualitative observations or scores (e.g., frailty based on walking patterns or walking patterns assessed based on height and/or weight) and/or other observations input by a practitioner or patient (e.g., using EMR and/or interfaces). A higher fall risk score may indicate a higher likelihood that a patient will fall or lose balance, or a higher frailty of the patient, but aspects disclosed herein are not limited.

The joint stiffness score may be calculated based on bone imaging, kinematics (e.g., how quickly a patient can bend a joint), range of motion, alignment, etc. Each joint (e.g., knee, hip, ankle, neck) may have its own joint stiffness score. A higher joint stiffness score may mean a higher stiffness and/or less laxity at the joint, but aspects disclosed herein are not limited.

The push-off power score may be based on kinematics, such as measured force, acceleration, contact pressure, etc. at a foot during walking (e.g., from a sensor in a shoe, coupled to the shoe, or coupled to the leg). A higher push-off power score may indicate a faster or stronger push-off during walking or spring in a step. Alternatively or in addition thereto, the push-off score may be measured at the hands, such as during push-ups.

The fracture risk score may be calculated from kinematics, range of motion (e.g., postural sway), bone density, and alignment. The fracture risk score may be paired with or be calculated based on lifestyle data and/or the fall risk score. The fracture risk score may also be based on other preoperative outputs 7000 and/or qualitative observations or scores (e.g., frailty based on walking patterns or walking patterns assessed based on height and/or weight) and/or other observations input by a practitioner or patient (e.g., using EMR and/or interfaces). As an example, a lower bone density score and a higher fall risk score may result in a higher determined fracture risk score. A higher fracture risk score may indicate a higher likelihood that a patient will fracture a bone, or a higher frailty of the patient, but aspects disclosed herein are not limited. Any of the scores discussed herein, such as fall risk score or facture risk score, may be determined with data from a humanoid robot 120 configured to mimic one or more attributes of a patient. These attributes of the patient may be a shape of the patient's bones, a poster, a gate, a flexibility, leg length discrepancy, leg length offset, or any other attribute described herein.

Intraoperative data 2000 may include information taken during performance of a procedure plan. Intraoperative data 2000 may include information on operating room efficiency, procedure duration, tourniquet time, blood loss, biometrics, incision length, soft tissue integrity, pressure, range of motion or other kinematics, implant or prosthesis position, and implant type or design, though this list is not exhaustive. For example, intraoperative data 2000 may also include updated preoperative data 1000 (e.g., updated bone imaging, etc.).

Operating room efficiency may include procedure duration information 2020, a number of practitioners performing the procedure plan, a number of medical or surgical tools and/or robots used, etc. Operating room efficiency may also include information on an operating room layout, such as a room size, a setup, an orientation, starting location, and/or movement path of certain objects (e.g., humanoid robot 120, practitioner, surgeon or other staff member, operating room table, cameras, GUI, other equipment, or patient). Cameras and/or a navigational system may be used to track operating room efficiency and/or layout information. Operating room efficiency may include information on staff and/or surgeon's performing the procedure plan, experience of each staff member or surgeon, past surgeries performed by each staff member or surgeon, and also scheduling information in an institution (e.g., hospital, clinic, etc.) where the surgery is taking place. Operating room efficiency may also include information on ergonomics for each staff member or surgeon, such as movement and posture patterns (measured by, for example, wearable sensors, external sensors, cameras and/or navigational systems, humanoid surgical robot 120, etc.) System 20 may make determinations to optimize operating room efficiency. For example, based on ergonomics information, system 20 may determine that a table is too high for a robotic surgeon and determine a lower height for the table in an updated operating room layout to include in the procedure plan, which may increase operating room efficiency.

Procedure duration may include duration and/or other timing data of certain steps or procedures of the procedure plan and/or a total time of procedure plan. Tourniquet time may include a time a tourniquet, cuff, or other restrictive device is applied to a limb. In addition, tourniquet time information may include pressure information at specific times or for specific time periods, where pressure information may be pressure applied to the limb, blood pressure, and/or pressure of, for example, an inflatable tourniquet. Blood loss may include information on an amount of blood lost during performance of procedure plan. Biometrics may include all types of information included in preoperative biometrics and may also include other patient characteristics, such as temperature, heart rate, breathing rate, skin temperature, skin moisture, pressure exerted on the patient's skin, patient movement/activity etc. during performance of procedure plan, etc. Incision length may include a length, position, and/or number of incisions actually made during performance of procedure plan. Actual incision length may correspond to or be different from a predicted or planned incision length from data in planned procedure and/or procedure plan.

Soft tissue integrity may include structural, strength, or density information for muscles, tendons, ligaments, and/or other soft tissue structures (e.g., skin) of the patient. Soft tissue integrity may be based on observed injuries (e.g., Posterior Cruciate Ligament or PCL injuries) during performance of procedure plan and/or based on prior observations. Soft tissue integrity may be an input and/or an output based on other preoperative inputs 1000 and intraoperative inputs 2000. Soft tissue integrity may be determined from a laxity assessment where a physician may stress a joint to determine tissue integrity. The laxity assessment may be a manual and subjective process, or alternatively may be controlled and/or quantified with sensors (e.g., wearable sensors, sensored implants) to measure applied force and/or joint displacement. For example, a practitioner and/or humanoid robot 120 may perform a varus/valgus stress test on a knee where a controlled force is applied to a shank to assess collateral ligaments. Diagnostic imaging systems such as MRI scans may also be used to assess tissue integrity and/or to reveal structural or physiological changes. As another example, a practitioner may use a pendulum knee drop test (passive test) to determine overall stiffness or knee joint laxity. Soft tissue integrity may also be determined from bone density, which may be determined from diagnostic imaging systems, as bone density may be correlated to ligament integrity and/or soft tissue integrity.

Pressure may include information about a pressure or load (e.g., a contact pressure) applied to a patient's anatomy and/or a prosthetic component during performance of procedure plan. For example, pressure may include information on a magnitude and a position or center of a load applied to a prosthetic component or implant (e.g., humeral component, glenosphere component, tibia component, femoral component, etc.). Range of motion data may include similar information as preoperative range of motion, although a surgeon may be manipulating a patient's body instead of the patient manipulating his or her own body. Intraoperative range of motion may include manipulation under anesthesia (MUA) data based on movements, exercises, stretches, and/or other manipulation performed by the surgeon and/or humanoid robot 120 to assess movement, release pain, and break up scar tissue.

Implant position data may include information on an actual implant or alignment during performance of procedure plan. Actual implant position data may correspond to or be different from a predicted or planned implant position from data in planned procedure and/or procedure plan. Similarly, implant type data may include information on an actual implant type, design, material, etc. during performance of procedure plan. Actual implant type data may correspond to or be different from a predicted or planned implant type in planned procedure data and/or procedure plan data. Humanoid robot 120 may then measure and/or digitize the final implants to capture actual position data with one or more measurement systems, such as cameras, LIDAR, etc.

For example, a practitioner may record a different implant type used for a procedure that is different from planned implant type data.

Intraoperative data 200 may include any data generated during a medical procedure utilizing humanoid robot 120, including robotic test kinematics, robotic kinetics, computer vision, humanoid robot 120 activity, and humanoid robot 120 environment. The surgical plan may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000).

Robotic kinematics may include movement or position information at various areas or locations at an electromechanical system 125, and range of motion data. Additional robot kinematics data may include strength measurements and/or force measurements. For example, robotic kinematics may include data used to determine a push-off power, force, or acceleration, or a power, force, or acceleration at a toe during walking. Range of motion data may include a range of motion at one or more joints, such as an angular range or axes of joint motion, or flexion or extension data. For example, robotic kinematics may include a flexion value, where a flexion value of 180 degrees ±3 degrees may indicate a full extension of a joint, and any value other than 180 degrees ±3 degrees may indicate a joint in flexion where bones on either side of the joint intersect to form an angle other than 180 degrees. Some of this information may be estimated or determined based on raw data from motion sensor systems and/or other sensors on the programmable humanoid robot 120. For example, robotic kinematics may include how quickly the humanoid robot 120 can bend a joint, sit down, stand up, a push-off power during walking, etc. Robotic kinematics may also include steps (e.g., measured by a pedometer) and/or measured gait.

A user, such as a surgeon, may telemanipulate humanoid robot 120 with various control mechanisms. A user may utilize a virtual reality headset, an augmented reality headset, and/or a mixed reality headset, for example, to telemanipulate humanoid robot 120. For example, an augmented reality headset may track the user's spatial position, gaze, head tilt, hand positioning and movement, finger positioning and movement, and other metrics. The movements of the surgeon may thus be mapped to the movements of humanoid robot 120. For example, a user wearing an aforementioned headset may lift their arm, which may cause a corresponding movement of humanoid robot 120 in the surgery room. Similarly, a surgeon may be provided the view of humanoid robot 120 (e.g., a first person view) generated by imaging devices 124. In this way, the surgeon may visualize the surgical field with a similar field of view as an in-person surgery, such that the movements of the surgeon may be used to perform a successful medical procedure with humanoid surgical robot 120.

Intraoperative outputs 8000 may be determined via one or more intraoperative algorithms 5000. Intraoperative algorithms 5000 may also consider and/or analyze other previously stored data 50 of memory system 40 to determine intraoperative outputs 8000. Intraoperative outputs 8000 may include implant recommendations, component placement/alignment, soft tissue performance estimates, such as maximum weight limits and maximum ranges of motion, postoperative activity recommendations (type and duration), and implant design analytics, such as longevity of the implant, durability of the implant, etc.

Intraoperative outputs 8000 may include any data associated with a given medical procedure generated by any part, component, or element of environment 100, including any actions performed by a human or humanoid robot 120 associated with the given medical procedure. For example, intraoperative outputs 8000 may include a difference between an actual measurement and an expected measurement of a same portion of a procedure. For example, an expected measurement of a given cut may differ from the cut actually performed by humanoid robot 120. Another intraoperative output may include a difference between a motion of a surgeon and a corresponding motion by the humanoid robot 120. For example, a surgeon may move their arm a certain distance (as captured by an aforementioned headset or other control device), and humanoid robot 120 may move its arm less than the certain distance. One of ordinary skill in the art will appreciate that these differences may be accounted for and mapped to a given user, such that the user may control the scale of mapped movement. For example, the control or input to the mapped humanoid robot 120 may be adjusted based on one or more virtual scaling parameters which may be user specific. For example, a user may move their arm X units, and the humanoid robot 120 may move its arm 1.2X units (e.g., based on the scaling factor).

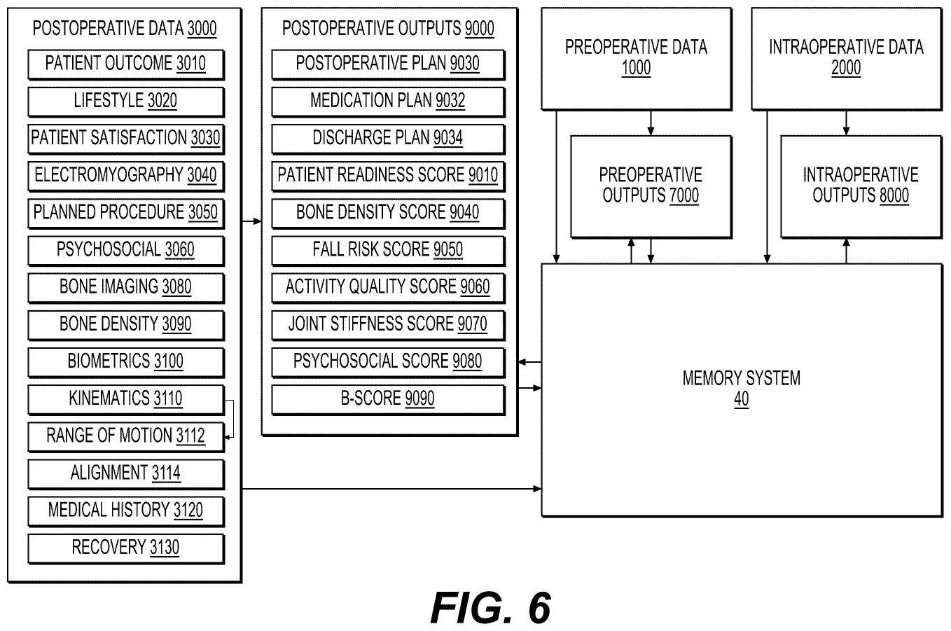

As shown in FIG. 6, intraoperative data 2000 and intraoperative outputs 8000 may be incorporated into determining postoperative outcomes. Postoperative data 3000 may include information on patient outcome 3010, lifestyle 3020, patient satisfaction 3030, electromyography (EMG) 3040, planned procedures 3050 (e.g., revisions), psychosocial 3060, bone imaging 3080, bone density 3090, biometrics 3100, and kinematics 3110 including range of motion 3112 and/or alignment 3114, postoperative medical history 3129, and recovery 3130. This list, however, is not exhaustive and postoperative data 3000 may include other patient specific information and/or other inputs manually input by a practitioner. Some of postoperative data 3000 may be directly sensed, and other postoperative data 3000 may be determined (e.g., using a postoperative algorithm 6000) based on directly sensed or input information.

Patient outcome 3010 may include both immediate and long term results and/or metrics from the medical procedure (e.g., surgery). For example, patient outcome 3010 may include a success metric or an indication of whether the procedure was successful, changes in range of motion, stability, fall risk or stability, fracture risk, joint stiffness or flexibility, or other changes between preoperative data 1000, or intraoperative data 2000 and postoperative data 3000, etc. Patient satisfaction 3030 may be a patient-reported (or, alternatively or in addition thereto, a practitioner-reported) satisfaction with the procedure, both immediate and long-term. Planned procedure 3050 may include information determined in outputting the postoperative plan and/or other information on future planned procedures for the patient (e.g., a surgeon-created plan or revision based on patient outcome 3010, etc.) Furthermore, humanoid PROMs information may be prompted given the designated phenotype to infer recovery trends or predict PROM information. Medical history 3120 may include updated and/or new medical history 3120 (as compared to preoperative medical history 1030) and may include both immediate and long term information such as new utilization of orthotics, care information in a supervised environment such as a skilled nursing facility or SNF, infection information, etc. Information on recovery 3130 may include information on adherence to a postoperative plan such as actual exercises performed, medicine dosage and/or type actually taken, fitness information, planned physical therapy (PT), adherence to PT, etc. Information on recovery 3130 may also include discharge and/or length of stay information.

Lifestyle 3020, EMG 3040, psychosocial 3060, bone imaging 3080, bone density 3090, biometrics 3100, kinematics 3110, range of motion 3112, and/or alignment 3114 may include similar types of information as preoperative lifestyle 1020, EMG 1040, psychosocial 1060, bone imaging 1080, bone density 1090, biometrics 3100, kinematics 1110, range of motion 1112, and alignment 1114. For example, psychosocial 3060 may include perceived pain, stress, happiness, anxiety, etc.

Postoperative outputs 9000 may be determined via one or more postoperative algorithms 6000. Postoperative algorithms 6000 may also consider preoperative information 1000 and/or outputs 7000, intraoperative data 2000 and/or outputs 8000, and/or other previously stored data 50 of memory system 40 to determine postoperative outputs 9000. Postoperative outputs 9000 may include an updated or new postoperative plan 9030, which may include a medication plan 9032 (e.g., for pain medication, antibiotics, etc.) and/or a discharge plan 9034, a patient readiness score 9010, an updated or new bone density score 9040, an updated or new fall risk or stability score 9050, an updated or new activity quality score 9060, an updated or new joint stiffness score 9070, an updated or new psychosocial score 9080, an updated or new B-score 9090, an updated or new push-off power score, and an updated or new fracture risk score. This list is not exhaustive, however. Updated or new bone density score 9040, fall risk or stability score 9050, activity quality score 9060, joint stiffness score 9070, psychosocial score 9080, B-score 9090, push-off power score 9100, or fracture risk score 9140 may include any of the features of preoperatively and intraoperatively determined data. Patient readiness score 9010 may indicate a readiness to be discharged (rather than a readiness for surgery) and may be based on (and updated using) postoperative data and outputs 3000, 9000, such as patient outcome 3010, lifestyle 3020, patient satisfaction 3030, electromyography 3040, psychosocial 3060, bone imaging 3080, biometrics 3100, kinematics 3110, recovery 3130, fall risk score 9050, activity quality score 9060, psychosocial score 9080, push-off power score 9100, fracture risk score 9140, etc.

As previously mentioned, postoperative algorithms 6000 may be used to output postoperative plan 9030. This postoperative plan 9030 may be newly generated based on postoperative data 3000 and/or may be a modification to postoperative plan 9030 generated using intraoperative data 2000 (and/or a manually input) and/or the postoperative plan 9030 generated using preoperative data 1000 (and/or manually input). In this context, for example, a medical practitioner may manually input an adjustment to postoperative plan 9030 via an electronic device. Postoperative plan 9030 may be continuously adjusted and/or updated as more postoperative data 3000 is collected.

As an example, postoperative algorithm 6000 may determine that only minor adjustments are necessary to update postoperative plan 9030 based on postoperative data 1000 like recovery 3130, kinematics 3110, biometrics 3100, patient satisfaction 3030, lifestyle 3020, etc. As another example, unexpected responses or conditions indicated by postoperative data 3000, which may differ from expected or optimized postoperative conditions (e.g., increased or decreased perceived pain, lower or higher range of motion 3112, unexpected injury indicated in medical history 3120, etc.), may be analyzed and considered, and postoperative algorithm 6000 may generate a new postoperative plan 9030 (e.g., based on stored data 50 from other patients with similar unexpected conditions).

Postoperative plan 9030 may include any of the features of the prehabilitation plan or procedure plans, such as an exercise program configured to decrease a recovery time of the patient. Postoperative plan 9030 may include a medication plan 9032 (e.g., pain medication plan including a type, dosage, and/or tapering of pain medication) and/or a discharge plan 9034 including a length of stay in a hospital. Medication plan 9032 may be based on psychosocial information 3060, and may further be based on biometrics 3100 (e.g., heart rate variability and/or sleep patterns).

Postoperative plan 9030 may include any of the features of the preoperatively determined postoperative plan, and may include a schedule of follow-up visits with a practitioner, surgeon, physical therapist, etc. Medication plan 9032 may include instructions for pain medication or other medication (e.g., antibiotics). For example, medication plan 9032 may include a medication type, active ingredient, mechanism of action, route of administration, dosage level, dosage plan (e.g., taper plan of dosing), frequency, and/or other instructions related to taking medication. Medication plan 9032 may be based on postoperative data 3000 and postoperative outputs 9000. For example, medication plan 9032 may be based on a patient's prior drug history, perceived pain and/or PROMS, biometrics 3100 like heart rate variability and sleep patterns, bone imaging 3080 (e.g., fractures or healing of fractures), infections or sickness (e.g., detected from changes in synovial fluid using sensored implants, detected from sensors measuring blood glucose, body temperature, sleep disturbances, heart rate variability, etc.) and other recovery 3130 data.

Medication plan 9032 may be updated continuously and/or periodically postoperatively. For example, biometrics 3100 like certain heart rate variability patterns (e.g., higher heart rate) and/or short or infrequent sleeping patterns may indicate that the patient is experiencing a higher level of pain, and medication plan 9032 may be updated to increase a dose or determine a different (or stronger) type of pain medication. As another example, sensored implants may detect information related to infections, and medication plan 9032 may be updated to include an antibiotic or other type of medication meant to treat the infection. Infection information may be sensed by sensored implants 316A or other sensors configured to detect a change in synovial fluid and configured to detect other biometrics 3100 such as heart rate variability, blood glucose, sleep disturbances, and body temperature which may indicate an infection at the surgical site. As another example, addictive behaviors may be determined (e.g., using biometrics 3100 and EMG data 3040 in combination with patient medical history or lifestyle 1020 and/or 3020 and/or other PROMS data), and medication plan 9032 may be created or updated to avoid and/or taper addictive pain medication, like opioids.

Discharge plan 9034 may include instructions for immediate recovery after surgery, such as a length of a hospital stay, supervision instructions, physical therapy instructions, target outputs (e.g., a fall risk threshold or target for fall risk score 9050, a target activity quality threshold or target for activity quality score 9060, a target patient readiness score 9010, a push-off power threshold or garget for push-off power 9100, and/or a fracture risk threshold or target for fracture risk score 9140), etc. Discharge plan 9034 may be based in preoperative, implant test, and postoperative data and outputs 1000, 2000, 3000, 7000, 8000, an/or 9000. For example, discharge plan 9032 may be based on recovery 3130, medical history 3120, patient satisfaction 3030, patient outcome 3010, bone imaging 3080, kinematics 3110, biometrics 3100, fall risk score 9050, activity quality score 9060, bone density score 9040, patient readiness score 9010, push-off power 9100, and/or fracture risk score 9140.

For example, discharge plan 9034 (and/or the patient readiness score 9010) may be updated using and/or based on postoperatively determined fall risk or stability score 9050 and/or postoperatively determined fracture risk score 91. Fall risk or stability score 9050 may be determined and/or updated using kinematics 3110 and biometrics 3100. Fracture risk score 9140 may be determined using fall risk score 9050 (or any inputs used to calculate fall risk score 9050) and bone density data 3090 and/or a determined bone density score 9040. Fall risk score 9050 and/or fracture risk score 9140 may increase, for example, based on certain (e.g., increased) heart rate combined with exit events and other kinematics data 3110 (e.g., acceleration data) from sensored implants 216A. Based on an increased fall risk score 9050 and/or fracture risk score 9140, discharge plan 9034 may be updated to increase a number of days in the hospital.

Algorithms 4000, 5000, and 6000 described herein may be further trained and/or refined over time for the instant patient and/or future patients based on all data 1000, 2000, and 3000 and outputs 7000, 8000, and 9000 may be stored in memory system 40, including patient outcome 3010 data. For example, algorithms 4000, 5000, and 6000 may learn and/or determine relationships across various data and parameters and make determinations (e.g., for an implant design or tightness, for exercises to include in pre or postoperative exercise plans, for medication plans, length of stay, etc.) based on those new learned, trained, and/or determined relationships.

For an instant patient, system 20 may use multiple preoperative algorithms 4000, intraoperative algorithms 5000, and postoperative algorithms 6000 prior to, during, and after a medical procedure to continuously monitor and track the patient and update or refine treatment and future humanoid robotic procedures.

The intraoperative data 2000 and postoperative data 3000 collected during and after procedures performed by humanoid robot 120 may be used to further train and refine the surgical phenotype selection module 121, including user model 1210 and surgical model 1212. For example, intraoperative outputs 8000 such as differences between expected and actual measurements, force and pressure data from sensors 123, and kinematic data from electromechanical systems 125 may be fed back into surgical model 1212 to improve the accuracy and precision of future robotic movements. Postoperative outcomes 9000, including patient outcome 3010, recovery 3130, and various scores such as fall risk score 9050 and activity quality score 9060, may be correlated with specific surgical techniques and phenotypes used by humanoid robot 120 to identify optimal approaches for different patient populations. This continuous feedback loop may enable humanoid robot 120 to learn from each procedure and progressively improve surgical performance, adapting to variations in patient anatomy, tissue characteristics, and procedural complexities over time.

FIG. 7 illustrates an exemplary testing and simulation diagram for surgical phenotype selection, as part of a humanoid surgical robot procedure. Surgical phenotype selection is an AI-based surgical algorithm to generate intraoperative outputs 8000 as described below. Stored data 1340 in memory system of FIG. 1 and/or stored data 50 in memory system 40 of FIG. 5 may be transmitted to system 20 and/or surgical phenotype selection module 121. Surgical phenotype selection may be determined at surgical phenotype selection module 121, or as an intraoperative algorithm 5000 performed at system 20.

Advantageously, a programmable humanoid robot 120 can be altered to perform tasks in a manner based on a human surgeon. The different types of surgeon styles and preferences are referred to as different surgical phenotypes, and the surgical phenotype selection module 121 can tune humanoid robot 120 to a particular surgical phenotype based on the given surgeon. Such an arrangement may be advantageous because multiple surgeons with unique and individualized preferences and techniques may be able to use a single humanoid robot 120. Surgical phenotype selection module 121 may receive input information 10 to determine a surgical phenotype selection.

As discussed above with reference to FIG. 4, input information 10 may include preoperative data 1000, intraoperative data 2000, and post-operative data 3000. System 20 may perform a plurality of algorithms, such as preoperative algorithms 4000, intraoperative algorithms 5000, and postoperative algorithms 6000 to generate output information 30. Output information 30 may include preoperative outputs 7000, intraoperative outputs 8000, and postoperative outputs 9000. Some or all of preoperative outputs 7000, intraoperative outputs 8000, and postoperative outputs 9000 may include determinations such as guidance for medical procedures, guidance for pre-operative or pre-habilitation treatment plans, guidance for post-operative or recovery plans, etc., as will be described in more detail hereinafter. System 20 may include one or more algorithms or modules configured to aggregate results from multiple preoperative algorithms 4000, intraoperative algorithms 5000, and/or postoperative algorithms 6000 to compile algorithm determinations for certain outputs (e.g., surgical plans, medical treatment plans, or instructions). As shown by the arrows in FIG. 4, preoperative outputs 7000, intraoperative outputs 8000, and postoperative outputs 9000 may become inputs into system 20 and/or memory system 40.

For example, from EMR data from preoperative data 1000, a patient's gender, age, body mass index (BMI), and joint line convergence angle (JLCA) may be determined. From the psychosocial scores and preoperative outputs 7000, a patient's pain scores, mobility issues, and risk of readmission, for example, may be determined. Other data and outputs described above may also be used in surgical phenotype selection. In the example shown in FIG. 7, four surgical phenotypes are shown: FP1, FP2, MP1, MP2. Surgical phenotype FP1 corresponds to a female surgeon with an individualized preferred alignment strategy, a pre-resection workflow type, a robotic user type, a medial parapatellar surgical approach, selective release of tight tissue soft tissue management style, and a patellar management style of resurfacing. Based on a series of test simulations, different surgery plans may be suggested, and different postoperative outcomes tracked. For example, in some cases, a first surgery plan M1 is suggested for the surgeon with surgical phenotype FP1. In other cases, a second surgery plan M2 is suggested for the surgeon with surgical phenotype FP1. M1 and M2 may differ in terms of, for example, alignment, component type, component placement, etc. Furthermore, the subset of those surgeries that resulted in “excellent,” “good,” or “” ad” postoperative outcomes are tracked and recorded as part of postoperative data 8000.

In the example shown in FIG. 7, subset 701 of FP1 cases resulted in an excellent postoperative outcome after an M1 surgery, subset 702 resulted in a good postoperative outcome after an M1 surgery, and subset 703 resulted in a bad outcome after an M1 surgery. Furthermore, subset 704 of FP1 cases resulted in an excellent postoperative outcome after an M2 surgery, subset 705 resulted in a good postoperative outcome after an M2 surgery, and subset 706 resulted in a bad outcome after an M2 surgery. Similar postoperative outcomes are measured for the remaining surgical phenotypes, and the postoperative outcomes may be included in the postoperative data 8000 that may be used to further tune surgical phenotype selection module 121 for humanoid robot training and optimization. These outcomes enable continuous refinement of the telemanipulated surgical robot's performance based on the specific surgical phenotype characteristics, allowing the humanoid robot 120 to adapt its techniques and approaches to match the preferred methods of different surgeons.

Real-time feedback during telemanipulation of the humanoid robot 120 may be captured through force sensors, torque sensors, and haptic feedback systems integrated into the humanoid robot 120, allowing the system to record the precise forces and pressures applied by the humanoid robot 120 during various surgical tasks. This force and haptic data may be correlated with visual feedback from imaging devices 124 to create a comprehensive record of humanoid robot 120 interactions with patient anatomy and surgical instruments.

In some aspects, a telemanipulation interface may include wearable sensors that track surgeon hand movements, finger positions, and grip patterns during a surgical procedure, which may be analyzed to identify characteristic manipulation techniques associated with successful outcomes. The system 20 may compare the intended movements commanded by the surgeon through the telemanipulation interface with the actual movements executed by the humanoid robot 120, identifying any discrepancies that may indicate areas for improvement in the robot's response characteristics or control algorithms.

Intraoperative feedback may include real-time monitoring of tissue response to robotic manipulation. Sensors 123 on humanoid robot 120 may detect subtle variations in tissue resistance and compliance that may not be apparent through visual observation alone, providing quantitative data that may be associated with specific surgical phenotype selections and techniques.

Postoperative feedback may be integrated into the surgical phenotype refinement process by correlating specific telemanipulation patterns and robotic execution characteristics with patient outcomes 3010. For example, the system may analyze relationships between the force profiles used during bone preparation, the precision of implant placement as executed by humanoid robot 120, and subsequent patient recovery metrics such as range of motion 3112, pain levels from psychosocial data 3060, and functional outcomes from kinematics 3110.

The surgical phenotype selection module 121 may apply machine learning algorithms to identify patterns in the feedback data that distinguish successful surgical approaches from less optimal ones. For example, the module 121 may detect that certain force application patterns during soft tissue management correlate with reduced postoperative complications, or that specific sequencing of surgical steps performed by humanoid robot 120 under telemanipulation leads to improved alignment outcomes.

Feedback from multiple procedures performed by the same surgeon may be aggregated to create a refined surgical phenotype profile that captures not only the surgeon's preferred techniques but also the optimal execution parameters for humanoid robot 120 when replicating those techniques. This aggregated feedback may include statistical distributions of force applications, movement velocities, approach angles, and timing patterns that characterize the surgeon's style.

FIG. 8 shows an example machine learning training flow chart 800, according to some embodiments of the disclosure. Referring to FIG. 8, a given machine learning model is trained using training flow chart 800. The training data 810 includes one or more of stage inputs 812 and known outcomes 814 related to the machine learning model to be trained. Stage inputs 812 are from any applicable source, including text, visual representations, data, values, video representations, comparisons, and stage outputs, e.g., one or more outputs from one or more steps from FIG. 3. Known outcomes 814 are included for the machine learning models generated based on supervised or semi-supervised training or can be based on known labels, such as review classification labels. An unsupervised machine learning model is not trained using known outcomes 814. Known outcomes 814 include known or desired outputs for future inputs similar to or in the same category as stage inputs 812 that do not have corresponding known outputs.

Training data 810 and a training algorithm 820, e.g., one or more of the modules implemented using the machine learning model or used to train the machine learning model, are provided to a training component 830 that applies the training data 810 to training algorithm 820 to generate the machine learning model. According to an implementation, training component 830 is provided with comparison results 816 that compare a previous output of the corresponding machine learning model to apply the previous result to re-train the machine learning model. Comparison results 816 are used by training component 830 to update the corresponding machine learning model. Training algorithm 820 utilizes machine learning networks or models including, but not limited to, deep learning networks such as Deep Neural Networks (DNN), Convolutional Neural Networks (CNN), Fully Convolutional Networks (FCN), and Recurrent Neural Networks (RNN), transformers, probabilistic models such as Bayesian Networks and Graphical Models, classifiers such as K-Nearest Neighbors, or discriminative models such as Decision Forests and maximum margin methods, the model specifically discussed herein, or the like.

The machine learning models used herein are trained or used by adjusting one or more weights or one or more layers of the machine learning model. For example, during training, a given weight is adjusted (e.g., increased, decreased, removed) based on training data or input data. Similarly, a layer is updated, added, or removed based on training data or input data. The resulting outputs are adjusted based on the adjusted weights or layers.

The initial training of the machine learning models may be completed by utilizing data that has been tagged. In some embodiments, this tagged data serves as an input for supervised, imitation, reinforcement, and/or semi-supervised learning approaches. The tagging process can be done manually or automatically, depending on the desired level of accuracy and available resources.

Manual tagging involves human annotators who examine training data and assign appropriate classification labels based on the content and context of the training data. This method can yield high-quality labeled data, as humans can understand nuances and contextual information better than automated algorithms. However, manual tagging can be time-consuming and labor-intensive, especially when dealing with large datasets.

Automatic tagging, on the other hand, involves using algorithms, such as natural language processing techniques or pre-trained machine learning models, to assign classification labels to reviews. This approach is faster and more scalable than manual tagging but may not be as accurate, particularly when dealing with complex or ambiguous items. To improve the accuracy of automatic tagging, it can be combined with manual tagging in a semi-supervised learning approach, where a smaller set of manually tagged data is used to guide the automatic tagging process.

The data collection process can be done manually or using web-scraping techniques. Manual data collection can be time-consuming and may not cover all the available data sources. Web-scraping techniques, on the other hand, use automated tools and scripts to extract data from various sources, making the process faster and more comprehensive.

Once data has been collected and tagged with appropriate classification labels, it can be used as input for the machine learning model's training process. The model will learn to recognize patterns and features in the data that correspond to specific contexts for data. With sufficient training and accurate labeled data, the machine learning model can become adept at identifying context-specific outputs, enabling an efficient and effective model.

As one skilled in the art will appreciate in light of this disclosure, certain embodiments may be capable of achieving certain advantages, including some or all of the following: (1) improving the functionality of a computing system through a more streamlined communication interface for interacting with a display device; (2) improving the user experience in interacting with a computer system by providing the streamlined communication interface receiving dynamic and interactive information; and (3) improving the reliability of information in a database by using machine learning techniques to personalize a user experience.

It should be understood that embodiments in this disclosure are exemplary only, and that other embodiments may include various combinations of features from other embodiments, as well as additional or fewer features.

In general, any process or operation discussed in this disclosure that is understood to be computer-implementable may be performed by one or more processors of a computer system, such any of the systems or devices in the environment 100 of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

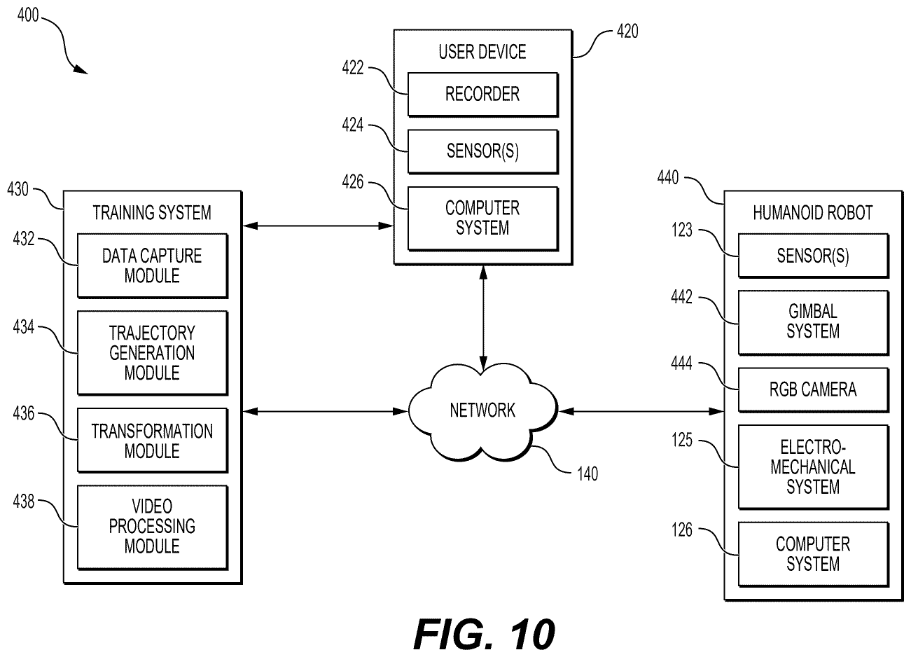

A computer system, such as a system or device implementing a process or operation in the examples above, may include one or more computing devices, such as one or more of the systems or devices in FIG. 1 or FIG. 10 to follow. One or more processors of a computer system may be included in a single computing device or distributed among a plurality of computing devices to perform a computer-implemented method. A memory of the computer system may include the respective memory of each computing device of the plurality of computing devices.

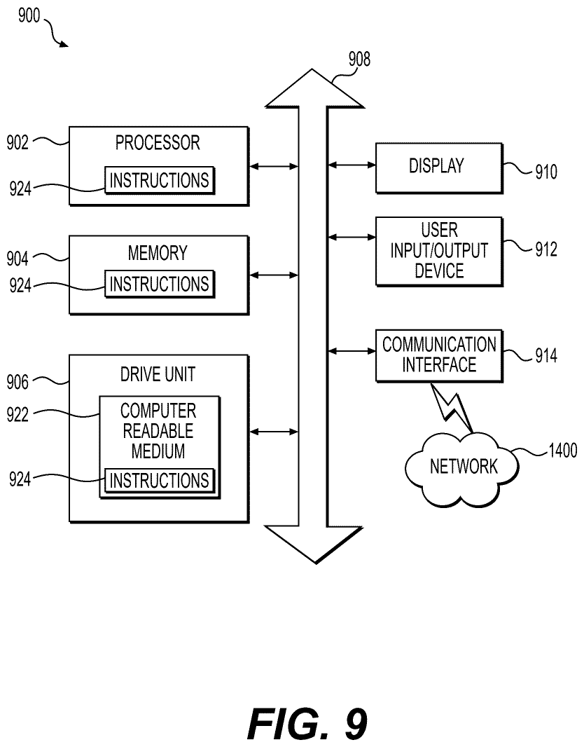

FIG. 9 is a simplified functional block diagram of a computer 900 that may be configured as a device for executing the methods described herein according to exemplary embodiments of the present disclosure. In various embodiments, any of the systems herein may be a computer 900 including, for example, a data communication interface 128 for packet data communication. Computer 900 also may include a central processing unit (“CPU”) 902, in the form of one or more processors, for executing program instructions. Computer 900 may include an internal communication bus 908, and a storage unit 906 (such as ROM, HDD, SDD, etc.) that may store data on a computer readable medium 922, although computer 900 may receive programming and data via network communications (e.g., via network 140). Computer 900 may also have a memory 904 (such as RAM) storing instructions 924 for executing techniques presented herein, although instructions 924 may be stored temporarily or permanently within other modules of computer 900 (e.g., processor 902 or computer readable medium 922). Computer 900 also may include input and output ports 912 or a display 910 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “items” or “articles of manufacture” typically in the form of executable code or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Aspects disclosed herein may be implemented during a robotic medical procedure where a robotic device, such as a surgical robot, a robotic tool manipulated or held by the surgeon and/or surgical robot, or other devices configured for automation perform at least a portion of a surgical procedure, such as a joint replacement procedure involving installation of an implant. Robotic device refers to surgical robot systems and/or robotic tool systems, and is not limited to a mobile or movable surgical robot. For example, robotic device may refer to a handheld robotic cutting tool, jig, burr, etc.

Any of the data collected using a humanoid robot described herein, and any additional data stored in an accessible database, may be incorporated into any of the algorithms and/or other processes described herein, and may be used to optimize algorithms using data collected from a humanoid robot 120 with a variety of different settings, such as range of motion, height, flexibility, and/or any other parameter described herein. Any parameter related to the humanoid robot 120 or the medical devices may be considered associated with the medical device (e.g. prosthetic element).

Humanoid robots 120 may be trained to perform various tasks through machine learning models that are trained on human demonstrations. A method for generating training data for training a humanoid robot 120 to perform a task may involve capturing and processing human performance data to create suitable training datasets. The captured human task performance data may be transformed into robot experience training data that can be utilized by machine learning models, as discussed in FIGS. 10-12.

FIG. 10 depicts an exemplary training environment 400 for training and using humanoid robots 120, according to aspects of this disclosure, to learn to perform tasks based on a human demonstration of a performance of the task. One or more components of the environment 400 may communicate with one or more of the other components of the environment 400 across electronic network 140, including one or more systems and elements associated with a user device 420, one or more systems and elements associated with a training system 430, which may be stored within a cloud, where the cloud may be any local or networked system suitable for transferring data, and one or more systems and elements associated with a programmable humanoid robot 440 Programmable humanoid robot 440 generally corresponds to programmable humanoid robot 120, described with reference to FIGS. 1-7, and may include all elements thereof. Programmable humanoid robot 440 may include other elements described herein, which may likewise be incorporated into programmable humanoid robot 120 described above.

The user device 420 may be configured to generate recording data of a user performing a task. For example, the user device 420 may be used to record a surgeon performing a surgery. In some examples, the user device 420 may be an extended-reality (“XR”) device worn as a headset by a user configured to generate a first-person, egocentric point of view recording of the arms and hands of the user while the user is performing a task, with the user device 420 being positioned at the head of the patient. The XR device may thus capture the human task performance in an egocentric view, allowing for comprehensive observation and recording of human movements during execution of the task. The user device 420 may be configured to capture visual information and inertial measurements of the upper body movements of a user performing the task, particularly the arms and hands of the user. The user device 420 may include a video recorder 422 for generating video image data of the user performing the task, one or more sensors 424 for generating position and movement data of the head of the user, and a computer system 426 for processing and analyzing the data generated by the video recorder 422 and the one or more sensors 424 to augment the data. Augmenting the data may include, for example, associating a time stamp with each of one or more video frames captured by the video recorder, and analyzing the images in each of the one or more video frames and successive video frames to determine position and trajectory data regarding the arms and hands of the user. Each video frame may be associated with a pose of the user's head, based on the IMU measurements from sensors 424, and the pose of the user's hands and arms that are visible in the frame.

The video recorder 422 may be a front-facing camera, such as a visible spectrum camera, that records a first person view in real time in color in the visible spectrum, commonly referred to as an “RGB” camera for “red, green, and blue,” the primary colors of the visible spectrum. The video recorder 422 may record, for example, the arms and hands of a user performing a task, such as a surgical procedure, when the user is wearing the user device 420 as a headset.

The sensors 424 within the user device 420 may include an IMU for tracking movement of the user's head, including position, velocity, acceleration, and trajectory data of the user's head. The IMU may include, for example, three gyroscopes and three accelerometers, where a first, second, and third gyroscope and a first, second, and third accelerometer are respectively aligned to three perpendicular axes. Each gyroscope may measure an angular velocity corresponding to a rotation about an axis. In other examples, the IMU may include any number of gyroscopes and any number of accelerometers, may only include one or more gyroscopes and not include accelerometers, or may only include one or more accelerometers and not include gyroscopes. Each accelerometer may measure a change in motion (acceleration) corresponding to one of the axes. The IMU may include up to nine degrees of freedom (DOF), which may include accelerations, gyroscopic velocities, and magnetometer values for 3-dimensional space. For example, the IMU may include up to 9-DOF, 6-DOF, or 3-DOF, and is not limited to the above-described arrangement. IMU 220 is configured to measure 6 degrees of freedom comprising translation movement along the X axis, Y axis, and Z axis as well as rotational movement such as yaw, roll, and pitch around each axis.

The IMU may include one or more micro-electro mechanical (MEMs) integrated circuits. For example, one or more of the gyroscopes or accelerometers may be or include a MEMs integrated circuit. The MEMs gyroscope may have a resonating mass that shifts with angular velocity and output a signal corresponding to (e.g., proportional to) the angular velocity of the IMU. A MEMs accelerometer may have a mass-spring system that shifts in response to an exerted acceleration, e.g., counter to a bias of a spring in the mass-spring system.

The computer system 426 may include software algorithms that are configured to track the location and orientation of the user's head from data generated by the sensors 424 and the user's arms and hands from data generated by the video recorder 422. The computer system 426 may include software algorithms for computing six degrees of freedom at each finger joint and arm joint throughout the video recording. Additionally, the computer system 426 may include a clock or other timekeeping device that provides time stamp data to the video recording, and on-board memory to store the recording data. The software algorithms may be configured to identify joints on the user's arms and hands from the video recording images and to determine inertial and positional data of the user's arms and hands including positions, velocities, and accelerations based on the time stamp data. The computer system 426 may calculate trajectories of the user's arms and hands based on the positions, velocities, and accelerations of the joints, such as the elbow, wrist, and finger and thumb joints.

Time stamps may be synchronized across the video recorder 422 and sensors 424 to ensure that each data point collected from the video recorder 422 and sensors 424 are correctly associated with each other. The pose data from the head tracking IMU and image data for the arms and hands may be saved at twice the frame rate of the video recorder 422.

The robotic training system 430 may comprise one or more modules configured to receive data from the user device 420 and to generate a robot experience video for use in training a humanoid robot to perform the task performed by the user in the video recording generated by the user device 420. The robotic training system may include a data capture module 432 configured to capture a video recording of a user performing a task. The video recording may include image data of at least one of hand positions and arm positions of the user and time stamp data associated with the image data. The data capture module 432 may be configured to synchronize the collection of sensor data and provide time stamps for each observation during the recording process. In some cases, the data capture module may be configured to receive data from the user device 420.

The robotic training system 430 may further include a trajectory generation module 434 configured to generate recorded trajectory data of the user performing the task based on the image data and the time stamp data. The trajectory generation module 434 may analyze the captured image data and time stamp data to identify user joints and generate movement and position data for anatomical joints, particularly of the user's arms and hands. The trajectory generation module 434 may process hand and arm tracking data over time to define trajectories of human arms and hands while performing the task.

A transformation module 436 may be configured to transform the recorded trajectory data into predicted robot trajectory data based on a kinematical model of a robot. The transformation module 436 may be configured to generate multiple possible robot trajectories and assign a confidence value to each robot trajectory. The transformation module may account for differences between human and robot kinematic models by planning trajectories that move the robot from starting to ending points. Each trajectory with sufficient confidence may be used to generate alternate robot trajectories for a single human trajectory.

The robotic training system 430 may include a video processing module 438 configured to generate a robot experience training video by digitally removing the user from video frames and inserting the kinematic model of the robot into the video frames based on the predicted robot trajectory. The video processing module 438 may employ background subtraction approaches using kinematically correct digital twins or artificial intelligence-based style transfer approaches using generative AI models. The video processing module may composite kinematically correct digital robot twins or database photos of robots at different poses into the processed video frames.

The robotic training system 430 may be configured to output the robot experience training video to a programmable humanoid robot 440. The robotic training system 430 may include a central server to format and transmit the processed robot experience training video along with associated trajectory data for use in training machine learning models that enable robots to perform tasks based on human demonstrations.

The central server associated with training system 430 may be used to manage and synchronize content associated with humanoid robot 440, ensuring consistent and up-to-date information is transmitted to humanoid robot 440. The central server may include a central content management system, using a high-level web framework to manage data stored in a memory system of the training system 430, with content delivery optimized through a content delivery network.

The programmable humanoid robot 440 may include various mechanical and electronic components as described with reference to FIG. 1 configured to enable autonomous task performance based on the trained machine learning models. Robot 440 may further include a gimbal system 442. Gimbal system 442 of robot 440 may provide multi-axis rotation capability, allowing a sensor system of sensors 123 to maintain proper orientation and tracking during task execution. In some aspects, the gimbal system 442 may incorporate servo motors or stepper motors with position feedback sensors to ensure precise angular positioning.

The sensors 123 may be mounted on the gimbal system 442 of robot 440 and may include multiple sensors. In some cases, the sensor system may incorporate depth sensors to provide three-dimensional environmental mapping capabilities. The imaging device 124 may include or be replace by an RGB camera 444. The RGB camera 444 of robot 440 may feature adjustable focus and exposure settings to adapt to varying lighting conditions experienced during task performance.

Robot 440 may include articulated arm assemblies with multiple degrees of freedom to replicate human arm and hand movements. The arm assemblies may incorporate joint actuators positioned at shoulder, elbow, and wrist locations corresponding to human anatomical joint positions. Each joint of robot 440 may include position encoders and force sensors to enable closed-loop control and force feedback during manipulation tasks. The end effectors of robot 440 may be configured as multi-fingered hands 1235 (see FIG. 2) or specialized grippers depending on the specific task requirements. In some cases, the end effectors may include tactile sensors distributed across finger surfaces to provide grip force feedback and object texture recognition. The fingers 1235 of robot 440 may incorporate compliant materials or springs to enable adaptive grasping of objects with varying shapes and sizes.

Robot 440 may include a central processing unit, such as computer system 126, and associated computing hardware configured to execute the trained machine learning models in real-time. The computing system may incorporate graphics processing units or specialized neural processing units to accelerate inference operations during task execution. In some aspects, robot 440 may include wireless communication modules to enable remote monitoring and control capabilities. Additionally, while a single (e.g., only one) humanoid robot 440 is represented in FIG. 10, it is understood that environment 400 may include a plurality of humanoid robots 440 (e.g., a network of humanoid robots) without departing from the scope of this disclosure.

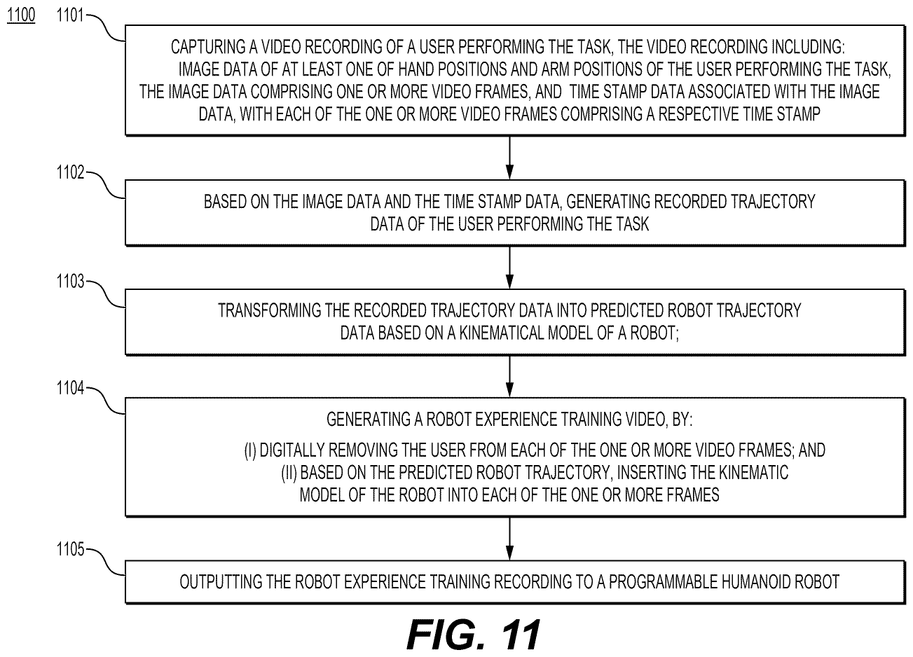

FIG. 11 is a flow chart showing a method 1100 for generating training data for training a humanoid robot to perform a task. At step 1101, the method 1100 includes capturing, by one or more processors, a video recording of a user performing a task. The video recording may include image data of at least one of hand positions and arm positions of the user performing the task, where the image data may comprise one or more video frames, and time stamp data associated with the image data, with each of the one or more video frames comprising a respective time stamp. In some cases, the video recording may be captured by an extended reality headset worn by the user performing the task. The extended reality headset may be configured for recording all data including RGB cameras, sensors for head tracking, and onboard processors for hand and arm tracking via the image data.

At step 1102, the includes generating, by the one or more processors, recorded trajectory data of the user performing the task based on the image data and the time stamp data. Generating the recorded trajectory data may comprise analyzing, by the one or more processors, the image data and the time stamp data to identify one or more user joints in the image data, and generating movement and position data for at least one of the one or more user joints based on the image data and the time stamp data. The extended reality headset may incorporate software algorithms that track six degree of freedom of poses at finger and arm anatomical joints. In some cases, poses from head tracking and hand/arm tracking may be saved at a rate that is twice the frame rate of the RGB cameras to better capture synchronization between the time stamp data and image data.

At step 1103, the method 1100 includes transforming, by the one or more processors, the recorded trajectory data into predicted robot trajectory data based on a kinematical model of a robot. Transforming the recorded trajectory data into predicted robot trajectory data may comprise generating multiple possible robot trajectories and assigning a confidence value to each robot trajectory. The system may generate multiple robot trajectories for a single human trajectory, with each trajectory assigned a confidence value to create alternate robot trajectories.

At step 1104, the method may proceed with generating, by the one or more processors, a robot experience training video. The robot experience training video may be generated by digitally removing the user from each of the one or more video frames and, based on the predicted robot trajectory, inserting the kinematic model of the robot into each of the one or more frames. Digitally removing the user from each of the one or more video frames may comprise a background subtraction approach that uses a kinematically correct digital twin of the user to create a mask for identifying regions of the video frames to be removed. The extended reality headset may incorporate stereo image recording capability that may be converted to a depth map for refining masks during human removal. In some cases, a background experience may be recorded by having the human keep their hands behind their back to capture the entire task region camera volume when suitable background pixels cannot be found.

In some cases, digitally removing the user from each of the one or more video frames may further comprise using an artificial intelligence-based style transfer approach that employs a generative artificial intelligence model trained to remove the user from the video frames. Robot insertion may be accomplished using a database of photos taken of the robot at different poses, where the trajectory pose may be mapped to the closest posed photo for compositing. A neural rendering AI may be used to post-process images to correct visual anomalies such as lighting and shadow effects in the resulting robot experience video. The system may be constructed end-to-end using a single generative AI model trained on multiple human/robot experience videos and trajectory sequences. The method 1100 may further include training a machine learning model using the robot experience training video to predict subsequent robot experience video frames based on prior robot experience video frames. At step 1105, the method 1300 may include outputting, by the one or more processors, the robot experience training recording to a programmable humanoid robot 440.

The robot experience training recording may be used to train the programmable humanoid robot 440 to perform tasks, such as surgical procedures. The trained machine learning model may enable the humanoid robot 440 to execute surgical tasks by processing real-time sensor data and generating appropriate motor commands based on patterns learned from the robot experience training video. During a surgical procedure, the humanoid robot 440 may capture current environmental data through its sensor system, including RGB camera data and inertial measurement data, and input this data along with predefined trajectory points into the trained machine learning model. The trained model may output predicted trajectory data for robot movement and predicted RGB frames, which may be used to generate robot motion commands for executing surgical tasks.

The robot experience training recording may enable the humanoid robot 440 to replicate surgical techniques and movements demonstrated by human surgeons in the original video recordings. The trained model may learn to recognize anatomical landmarks, surgical instruments, and appropriate manipulation techniques from the robot experience training video. In some cases, the humanoid robot 440 may perform consistency checking during surgical procedures by comparing predicted RGB frames generated by the trained model with subsequently captured RGB camera data to verify that the robot is performing the task correctly. When consistency checks indicate deviations from expected performance, the humanoid robot 440 may halt motion and compute corrective trajectories to maintain surgical precision.

The robot experience training recording may be integrated with the surgical phenotype selection module 121 to enable the humanoid robot 440 to adapt its surgical techniques based on specific surgeon preferences and styles. The trained machine learning model may learn to perform surgical tasks according to different surgical phenotypes by processing robot experience training videos generated from multiple surgeons with varying techniques and approaches. In some aspects, the humanoid robot 440 may use the trained model to perform portions of surgical procedures autonomously while maintaining the ability to receive real-time guidance and corrections from remote surgeons through teleoperation interfaces.

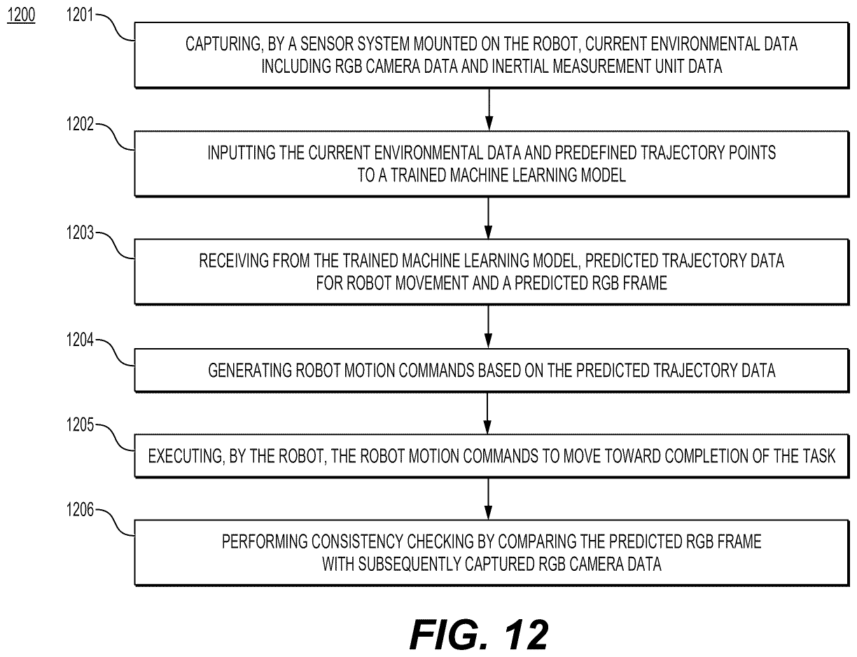

FIG. 12 is a flow chart showing a method 1200 of operating a trained humanoid robot to perform a task using the robot experience training video and trained machine learning models described above with reference to FIG. 11. At step 1201, the method includes capturing, by a sensor system mounted on the robot, current environmental data including RGB camera data and inertial measurement unit data. The sensor system may be mounted on a gimbal having at least three degrees of freedom. The gimbal may be positioned at a location corresponding to a human neck position relative to a shoulder. In some cases, the camera system may provide the same sensor package as the XR device used to collect human experience data in method 1100. When an XR device is used as the camera system for robot task execution, a synthetic human head may be mounted to the gimbal to which the XR device is attached.

At step 1202, the method 1200 may include inputting, by one or more processors, the current environmental data and predefined trajectory points for the robot arms and hands to a trained machine learning model. The trained machine learning model

may have been trained using robot experience training video generated by digitally removing a human user from human task performance video and inserting a kinematic model of the robot, as discussed above.

At step 1203, the method 1200 may include receiving, by the one or more processors, from the trained machine learning model, predicted trajectory data for robot movement and a predicted RGB frame. The trained machine learning model may output both the predicted trajectory for robot movement and the predicted RGB frame for the next trajectory point.

At step 1204, the method 1200 may include generating, by the one or more processors, robot motion commands based on the predicted trajectory data. The predicted trajectory may be submitted to a robot motion controller, as described above with reference to FIGS. 1-3, to initiate robot movement.

At step 1205 the method 1200 may include executing, by the robot, the robot motion commands to move toward completion of the task. The robot arm or arms may be sufficient in degrees of freedom and length to allow the robot to reach starting and ending trajectory points defined within the human experience data.

At step 1206, the method may include performing, by the one or more processors, consistency checking by comparing the predicted RGB frame with subsequently captured RGB camera data. Consistency frame checking may be performed on a regular interval that matches the frame rate of human experience video during robot task execution. Performing consistency checking may further comprise halting robot motion when the predicted RGB frame fails to match the subsequently captured RGB camera data within a predetermined threshold. In some cases, performing consistency checking may further comprise computing a new trajectory for a motion controller when the consistency checking fails. The robot may take corrective action by computing the new trajectory for the motion controller in addition to halting robot motion when consistency checks fail. In some examples, the robot experience training video may be generated based on a specific surgical phenotype determined by the surgical phenotype selection module 121, such that the trained machine learning model may learn to perform tasks according to the techniques, preferences, and movements associated with a particular surgeon or surgical approach.

The humanoid robot 440 may be trained to optimize operating room layout and equipment positioning based on learned patterns from the robot training videos and prior procedures. The humanoid robot 440 may analyze reachability and work volume requirements to determine optimal positioning within the operating room for different procedure types. In spine procedures involving long constructs, the humanoid robot 440 may reposition itself to maintain optimal access throughout the procedure. The humanoid robot 440 may also provide guidance for placement of navigation equipment, including optical camera systems, based on learned optimal configurations that maximize tracking accuracy and minimize interference with surgical workflow. The humanoid robot 440 may further approach the setup table to inspect instrument trays and implants, verifying that all required instruments and correct implant sizes and types are present before the procedure begins.

The humanoid robot 440 may be trained to identify optimal locations for anatomy trackers and guide surgeons in placing these trackers on bone with appropriate orientation relative to optical cameras for accurate tracking. For total knee arthroplasty procedures, the humanoid robot 440 may be trained to hold the patient's leg and perform pivot motions to compute hip center as a patient landmark. The humanoid robot 440 may also be trained to identify and touch off medial and lateral malleoli as patient landmarks for tibial registration. In some cases, the humanoid robot 440 may hold a sharp probe and capture registration points on bone surfaces, with the ability to detect cartilage using neural network models, such as those described with reference to FIG. 8 above, and estimate cartilage thickness to determine appropriate penetration depth for accurate point capture on underlying bone. Force-torque sensors in the end effectors of the humanoid robot 440 may provide input to neural network models to verify that registration points are being captured on bone rather than on cartilage or deep within bone.

The humanoid robot 440 may be trained to optimize probe orientation relative to optical tracking systems for accurate point capture and may employ control algorithms to maintain tool stability during registration procedures. Training on diverse patient anatomies may enable the humanoid robot 440 to perform patient-specific bone registration by selecting points in regions that lead to higher registration accuracy and precision. Improved point capture accuracy may reduce the number of registration points required for robust bone registration compared to manual techniques. The humanoid robot 440 may also be trained to complete bone registration verification procedures, with the potential to automate verification when quality metrics fall below defined thresholds.

The humanoid robot 440's vision system may employ deep learning pose estimation models to estimate the pose of anatomy trackers relative to anatomy coordinate systems following bone registration verification. During procedures, the humanoid robot 440 may be prompted via voice command to perform additional pose estimations that may be compared against initial poses to detect tracker movement. When differences between poses exceed predetermined thresholds, the humanoid robot 440 may issue warnings that bone registration may no longer be valid and may proceed to re-register upon surgeon confirmation. This approach may replace traditional checkpoint installation, capture, and verification processes across multiple procedure types including total hip arthroplasty, shoulder procedures, and spine procedures.

For shoulder and total hip arthroplasty procedures, the humanoid robot 440 may be trained to capture patient landmarks for glenoid and humeral registration using sharp probes. Alternatively, the vision system of the humanoid robot 440 may perform machine vision segmentation and keypoint detection to eliminate the need for probe-based landmark capture. For spine procedures, the humanoid robot 440 may be trained to capture registration points on each vertebra to register multiple spinal levels for accurate bone registration. The humanoid robot 440 may also be trained to assist with or completely perform joint balancing assessments in total knee arthroplasty, using its vision system to estimate lateral and medial gaps in extension and flexion in combination with feedback from tracking systems. Using this feedback, the humanoid robot 440 may apply learned force and torque patterns with both hands to perform trialing and identify optimal implant alignments, with the ability to adjust surgical plans through voice commands to target laxity windows and minimize soft tissue release requirements.

The humanoid robot 440 may be trained to perform bone resection and preparation procedures automatically or semi-automatically, with no-fly zones established based on planned resections from preoperative imaging. The humanoid robot 440 may be trained to detect soft tissue and critical anatomical structures, enabling it to avoid sensitive areas during bone preparation. For spine procedures, the humanoid robot 440 may perform drilling and tapping operations while tracking vertebrae at each level, adjusting its movements in response to detected anatomical movement. In some aspects, the dual-hand capability of humanoid robot 440 may enable simultaneous bone preparation on one side or level while placing screws on another side or level during spinal fusion procedures.

The humanoid robot 440 may assist with implant placement procedures, including acetabular cup impaction in total hip arthroplasty to press-fit the cup into the reamed socket, either automatically or semi-automatically. The humanoid robot 440's ability to perform these tasks may be based on training with multiple surgical demonstrations as described in FIGS. 10 and 11 above, and continuous refinement through feedback from actual procedures. The integration of vision systems, force-torque sensing, and machine learning models may enable the humanoid robot 440 to adapt its techniques to patient-specific anatomical variations while maintaining consistent execution of surgical steps. In some cases, the humanoid robot 440 may work collaboratively with surgeons, performing certain procedural steps autonomously while the surgeon maintains oversight and performs other portions of the procedure manually.

The standardization of surgical techniques through robotic execution may reduce procedural variability and potentially improve clinical outcomes, particularly for challenging aspects of procedures such as joint balancing in total knee arthroplasty. The humanoid robot 440 has an ability to learn from multiple surgical demonstrations and adapt to different surgical phenotypes, thus enabling the humanoid robot 440 to replicate the techniques of experienced surgeons while maintaining consistency across procedures. The continuous feedback loops between the humanoid robot 440 sensors, vision systems, and control algorithms may enable real-time adjustments during procedures to maintain accuracy and precision. As the humanoid robot 440 accumulates experience across multiple procedures and patient anatomies, its performance may continue to improve through ongoing machine learning model refinement and incorporation of new surgical techniques and approaches.

The systems and methods disclosed herein may provide improvements to both telemanipulated and autonomous surgical robots. The surgical phenotype selection module 121 may enhance telemanipulated surgical robot performance by enabling the humanoid robot 120 to adapt its mechanical and control parameters to match the specific techniques, preferences, and physical characteristics of individual surgeons. By adjusting parameters such as joint stiffness, range of motion, movement speed, and force application profiles based on user model 1210 and surgical model 1212, the telemanipulated humanoid robot 120 may replicate the natural working style of different surgeons, thereby improving surgical precision. The phenotype selection approach may allow a single humanoid robot 120 to serve multiple surgeons with varying surgical approaches, alignment strategies, and soft tissue management techniques, making the technology more accessible and adaptable across different surgical contexts and institutions.

Additionally, the use of extended reality devices to capture human task performance may provide improvements in training humanoid robots 120 by generating high-quality training data that preserves the spatial relationships and movement patterns of human surgical techniques. By recording surgical procedures from an egocentric perspective using RGB cameras and inertial measurement units, the system may capture detailed trajectory data of hand and arm movements along with corresponding visual context, which may then be transformed into robot experience training videos through digital removal of the human operator and insertion of the robot kinematic model. This approach may enable machine learning models to learn surgical tasks from human demonstrations while accounting for the kinematic differences between human anatomy and robotic systems, allowing the trained models to generate appropriate motor commands during autonomous task execution. The robot experience training videos may provide the trained machine learning models with visual and trajectory data that enables real-time consistency checking during surgical procedures, where predicted RGB frames may be compared with actual camera data to verify correct task execution and trigger corrective actions when deviations occur.

Feedback mechanisms from actual robot surgeries, whether performed through telemanipulation or autonomous execution, may provide continuous improvement opportunities for both operational modes by generating intraoperative data 2000 and postoperative data 3000 that may be analyzed to refine surgical phenotype selection parameters, update machine learning models, and optimize surgical planning algorithms. Intraoperative outputs 8000 such as force measurements, trajectory deviations, and tissue response data collected during robotic procedures may be fed back into the surgical phenotype selection module 121 to improve the accuracy of phenotype matching for telemanipulated robots and to generate additional training data for autonomous systems. Postoperative outcomes 9000 including patient recovery metrics, range of motion measurements, and complication rates may be correlated with specific surgical techniques and robot execution parameters to identify optimal approaches for different patient populations and anatomical variations. This continuous feedback loop may enable both telemanipulated and autonomous surgical robots to progressively improve their performance over time, adapting to variations in surgical complexity, patient anatomy, and procedural requirements while maintaining the ability to incorporate new surgical techniques and approaches as they are developed and validated through clinical practice.

Aspects disclosed herein are not limited to specific scores, thresholds, etc. that are described. For example, outputs and/or scores disclosed herein may include other types of scores such as Hoos Koos, SF-12, SF-36, Harris Hip Score, etc. Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes. Aspects disclosed herein may augment the continuum of care to optimize post-operative outcomes for a patient. Aspects disclosed herein may recognize or determine previously unknown relationships, such as how injuries or movement at one joint affects movement at a different joint, to help optimize care, such as placement, type, and/or design of a prosthetic.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A method for gathering surgical data, the method comprising:
programming a humanoid robot to perform a task, wherein the humanoid robot includes one or more measurement devices coupled to the humanoid robot; and
receiving surgical data associated with the one or more measurement devices during a performance of the task by the humanoid robot.

2. The method of claim 1, wherein the humanoid robot includes a surgical phenotype selection module, the method comprising:
determining a surgical phenotype of the humanoid robot based on the surgical phenotype selection module.

3. The method of claim 2, wherein the surgical phenotype selection module includes a user model, and the humanoid robot includes one or more electromechanical systems, the method further comprising:
actuating the one or more electromechanical systems to conform to the surgical phenotype determined by the surgical phenotype selection module, wherein the surgical phenotype is determined based on the user model.

4. The method of claim 3, wherein the user model is a trained machine learning model.

5. The method of claim 2, wherein the surgical phenotype selection module includes a surgical model configured to receive surgical data including at least one movement of a human surgeon performed during a surgery, and programming the humanoid robot to perform the task is based on an output from the surgical model.

6. The method of claim 5, wherein the surgical model is a trained machine learning model.

7. The method of claim 2, further comprising:
receiving surgical phenotype data from one or more data sources, the one or more data sources including prior surgical data of one or more patients with at least one attribute in common with a current patient; and
determining the surgical phenotype of the humanoid robot based on the phenotype data.

8. The method of claim 7, wherein the one or more data sources includes at least one of: preoperative data relating to one or more patients, postoperative data relating to one or more patient, and intraoperative data.

9. The method of claim 1, further comprising:
determining intraoperative outputs based on intraoperative data, the intraoperative outputs including one or more of: a difference between an actual measurement and an expected measurement associated with the task; or a difference between a motion of a surgeon and a corresponding motion by the humanoid robot.

10. The method of claim 9, wherein the intraoperative data is received from one or more sensors; the one or more sensors including at least one of: a force sensor, a displacement sensor, a pressure sensor, and an imaging device.

11. A system for gathering test data for one or more medical devices, the system comprising:
a humanoid robot comprising one or more processors;
one or more measurement devices coupled to the humanoid robot; and
one or more sensors configured to gather intraoperative data associated with the one or more measurement devices during a performance of a programmed task by the humanoid robot.

12. The system of claim 11, wherein the humanoid robot includes a surgical phenotype selection module configured to determine a surgical phenotype of the humanoid robot.

13. The system of claim 12, wherein the surgical phenotype selection module includes a surgical model configured to receive surgical data including at least one movement of a human surgeon performed during a surgery, and the humanoid robot includes one or more electromechanical systems, wherein parameters of the one or more electromechanical systems are defined based on the surgical phenotype selection module.

14. The system of claim 13, wherein the surgical model is a trained machine learning model.

15. The system of claim 12, wherein the surgical phenotype selection module includes a surgical model, and programming the humanoid robot to perform the task is based on output from the surgical model.

16. The system of claim 15, wherein the surgical model is a trained machine learning model.

17. The system of claim 12, further comprising:
one or more data sources comprising surgical phenotype data, wherein the surgical phenotype data includes both surgical model data and user model data, wherein determining the surgical phenotype of the humanoid robot is based on the surgical phenotype data.

18. The system of claim 17, wherein the one or more data sources includes at least one of: preoperative data relating to one or more patients, postoperative data relating to one or patients, or intraoperative data.

19. The system of claim 11, wherein the system is further configured to determine intraoperative outputs based on the intraoperative data, the intraoperative outputs including one or more of: a difference between an actual measurement and an expected measurement associated with the task; or a difference between a motion of a surgeon and a corresponding motion by the humanoid robot.

20. A system comprising:
a programmable humanoid robot including one or more measurement sensors;
wherein the programmable humanoid robot comprises:
at least one processor; and
at least one storage medium comprising instructions which, when executed by the at least one processor, cause the at least one processor to perform operations comprising:
receive surgical phenotype data from one or more data sources;
determine, using a surgical phenotype selection module, a surgical phenotype of the humanoid robot based on the surgical phenotype data;
adjust one or more electromechanical systems to conform the humanoid robot to the surgical phenotype determined by the surgical phenotype selection module;
program the humanoid robot to perform a task based on a surgical model;
receive intraoperative data associated with the one or more measurement sensors during a performance of the task by the humanoid robot; and
causing to display, on one or more display devices; intraoperative outputs based on the intraoperative data associated with the one or more measurement sensors.

21. A computer-implemented method for generating training data for training a humanoid robot to perform a task, comprising:
capturing, by one or more processors, a video recording of a user performing the task, the video recording including:
image data of at least one of hand positions and arm positions of the user performing the task, the image data comprising one or more video frames, and
time stamp data associated with the image data, with each of the one or more video frames comprising a respective time stamp;
based on the image data and the time stamp data, generating, by the one or more processors, recorded trajectory data of the user performing the task;
transforming, by the one or more processors, the recorded trajectory data into predicted robot trajectory data based on a kinematical model of a robot;
generating, by the one or more processors, a robot experience training video, by:
(i) digitally removing the user from each of the one or more video frames; and
(ii) based on the predicted robot trajectory, inserting the kinematic model of the robot into each of the one or more frames; and
outputting, by the one or more processors, the robot experience training recording to a programmable humanoid robot.

22. The method of claim 21, wherein the video recording is captured by an extended-reality (xR) headset worn by the user performing the task.

23. The method of claim 22, wherein the headseat includes an IMU for generating movement and position data for a head of the user.

24. The method of claim 21, wherein generating the recorded trajectory data comprises analyzing, by the one or more processors, the image data and the time stamp data to identify one or more user joints in the image data, and generating movement and position data for at least one of the one or more user joints based on the image data and the time stamp data.

25. The method of claim 21, wherein transforming the recorded trajectory data into predicted robot trajectory data comprises generating multiple possible robot trajectories and assigning a confidence value to each robot trajectory.

26. The method of claim 21, wherein digitally removing the user from each of the one or more video frames comprises a background subtraction approach that uses a kinematically correct digital twin of the user to create a mask for identifying regions of the video frames to be removed.

27. The method of claim 26, wherein digitally removing the user from each of the one or more video frames further comprises using a style transfer approach that employs a generative artificial intelligence model trained to remove the user from the video frames.

28. The method of claim 21, further comprising training a machine learning model using the robot experience training video to predict subsequent robot experience video frames based on prior robot experience video frames.

29. The method of claim 28, further comprising operating a robot to perform the task based on outputs of the trained machine learning model.

30. The method of claim 29, wherein the robot includes a gimbal and a sensor system attached to the gimbal, the sensor system including an RGB camera and an inertial measurement unit.

31. A robotic training system, comprising:
a data capture module configured to capture a video recording of a user performing a task, the video recording including image data of at least one of hand positions and arm positions of the user and time stamp data associated with the image data;
a trajectory generation module configured to generate recorded trajectory data of the user performing the task based on the image data and the time stamp data;
a transformation module configured to transform the recorded trajectory data into predicted robot trajectory data based on a kinematical model of a robot;
a video processing module configured to generate a robot experience training video by digitally removing the user from video frames and inserting the kinematic model of the robot into the video frames based on the predicted robot trajectory; and
an output module configured to output the robot experience training video to a programmable humanoid robot.

32. The robotic training system of claim 31, wherein the data capture module comprises an extended reality headset configured to be worn by the user.

33. The robotic training system of claim 32, wherein the extended reality headset includes an inertial measurement unit configured to generate movement and position data for a head of the user.

34. The robotic training system of claim 31, wherein the transformation module is configured to generate multiple possible robot trajectories and assign a confidence value to each robot trajectory.

35. A method of operating a trained humanoid robot to perform a task, comprising:
capturing, by a sensor system mounted on the robot, current environmental data including RGB camera data and inertial measurement unit data;
inputting, by one or more processors, the current environmental data and predefined trajectory points to a trained machine learning model;
receiving, by the one or more processors, from the trained machine learning model, predicted trajectory data for robot movement and a predicted RGB frame;
generating, by the one or more processors, robot motion commands based on the predicted trajectory data;
executing, by the robot, the robot motion commands to move toward completion of the task; and
performing, by the one or more processors, consistency checking by comparing the predicted RGB frame with subsequently captured RGB camera data.

36. The method of claim 35, wherein the sensor system is mounted on a gimbal having at least three degrees of freedom.

37. The method of claim 36, wherein the gimbal is positioned at a location corresponding to a human neck position relative to a shoulder.

38. The method of claim 35, wherein performing consistency checking further comprises halting robot motion when the predicted RGB frame fails to match the subsequently captured RGB camera data within a predetermined threshold.

39. The method of claim 38, wherein performing consistency checking further comprises computing a new trajectory for a motion controller when the consistency checking fails.

40. The method of claim 35, wherein the trained machine learning model was trained using robot experience training video generated by digitally removing a human user from human task performance video and inserting a kinematic model of the robot.
